(19) United States
(12) Patent Application Publication
(10) Pub. No.: US 2025/0235560 A1
(43) Pub. Date: Jul. 24, 2025

(54) ADENO-ASSOCIATED VIRUS POTENCY ASSAY AND USES THEREOF

(71) Applicant: ASTELLAS GENE THERAPIES, INC., South San Francisco, CA (US)

(72) Inventors: Yukari MORIYA, Tsukuba (JP); Tomonari WATABIKI, San Francisco, CA (US); Kenichiro ISHIZU, Tsukuba (JP); Toshiko ASANO, Tsukuba (JP)

(21) Appl. No.: 18/853,473

(22) PCT Filed: Apr. 5, 2023
(86) PCT No.: PCT/US2023/065389

§ 371 Date: Oct. 2, 2024

Related U.S. Application Data
(60) Provisional application No. 63/327,574, filed on Apr. 5, 2022.

(51) Int. Cl. A61K 48/00; C12N 9/16; C12N 15/10

(52) U.S. Cl. CPC A61K 48/0058; C12N 9/16; C12N 15/1082; C12Y 301/03

(57) ABSTRACT

Some embodiments relate to methods and kits for detecting and monitoring the potency of an enzyme encoded by an adeno-associated virus. Exemplary enzymes that may be used in conjunction with the compositions and methods of the disclosure are Myotubularin 1 (MTM1) and myotubularin-related proteins, such as myotubularin-related protein 1 (MTMR1), MTMR2, MTMR3, MTMR4, MTMR5, MTMR6, MTMR7, MTMR8, MTMR9, MTMR10, MTMR11, MTMR12, MTMR12, MTMR13, and MTMR14, among others.

### CROSS-REFERENCE TO RELATED APPLICATION(S)

This application is a national phase filing under 35 C.F.R. § 371 of and claims priority to PCT Patent Application No. PCT/US2023/065389, filed on Apr. 5, 2023, which claims the benefit of priority to U.S. Provisional Application Ser. No. 63/327,574, which was filed Apr. 5, 2022, the entire disclosure of which is hereby incorporated by reference as if set forth in its entirety.

### SEQUENCE LISTING

The instant application contains a Sequence Listing which has been submitted electronically in XML format and is hereby incorporated by reference in its entirety. Said XML copy, created on Mar. 30, 2023, is named “51037-061WO2_Sequence_Listing_3_30_23” and is 20,293 bytes in size.

### FIELD OF THE DISCLOSURE

The presently disclosed subject matter relates to adeno-associated virus (AAV) potency assays, and more particularly to methods, uses, and kits for monitoring the activity of an enzyme encoded by an AAV.

### BACKGROUND OF THE DISCLOSURE

One of the most important classes of intracellular activities is post-translational modification of proteins. Post-translational modifications effect changes in the biological activity and/or function of putatively modified proteins. Post-translational modifications can be effectuated by enzymes (e.g., phosphatases). For example, protein phosphatases, a class of enzymes, remove phosphate groups from proteins, and a change in the phosphorylation state of a protein can affect protein function, such as protein localization or protein-protein interactions. Such changes in protein function can subsequently modulate cellular metabolism, regulation, growth, differentiation, though in some cases the altered protein function is aberrant, leading to pathology.

Researchers have found many human diseases and disorders arising from genetic defects in protein phosphatases. For example, X-linked myotubular myopathy (XLM™) is a fatal monogenic disease of skeletal muscle, resulting from loss-of-function mutations in the protein phosphatase Myotubularin 1 (MTM1). Approximately one in every 50,000 newborn boys has XLM™, which typically displays as marked hypotonia and respiratory failure. Recently, viral vector-mediated (e.g., adeno-associated virus (AAV)-mediated) gene therapy approaches involving the delivery of MTM1 have been developed for the treatment of XLM™. However, there is a need in the art for improved methods of assessing the efficacy of viral vectors (e.g., AAVs) encoding enzymes (e.g., phosphatases), such as AAVs encoding MTM1, that are sensitive and adaptable to high-throughput screening methods.

### SUMMARY OF THE DISCLOSURE

The disclosure provides methods and uses for determining the efficacy of an adeno-associated virus (AAV) vector encoding an enzyme (e.g., Myotubularin 1 (MTM1)), for example by assessing a quantity or concentration of a product resulting from cleavage of a substrate by the enzyme by way of high-performance liquid chromatograph (HPLC) in an AAV-permissive cell, surrounding extracellular media, or from cell lysate. In some embodiments, after a change in the quantity or concentration of the cleavage product is determined, a patient is administered a therapeutically effective amount of the AAV encoding the enzyme.

In one aspect, the disclosure provides a method of determining the efficacy of an AAV vector encoding an enzyme for treatment of a loss-of-function disease associated with a deleterious mutation in a gene encoding the enzyme in a patient, the method including: a) contacting the AAV vector with an AAV-permissive cell expressing a cleavable substrate specific for the enzyme for a time sufficient for the AAV-permissive cell to express the enzyme; and b) assessing, by way of chromatography, a quantity or concentration of the substrate or of a product resulting from cleavage of the substrate by the enzyme in the cell or surrounding extracellular media, wherein a finding that (i) the quantity or concentration of the cleavage product in the cell or surrounding extracellular media is increased relative to a reference quantity or concentration of the cleavage product or (ii) the quantity or concentration of the substrate in the cell or surrounding extracellular media is decreased relative to a reference quantity or concentration of the substrate identifies the AAV vector as being efficacious for treating the disease.

In another aspect, the disclosure provides a method of determining the efficacy of an AAV vector encoding an enzyme for treatment of a loss-of-function disease associated with a deleterious mutation in a gene encoding the enzyme in a patient, the method including: a) contacting the AAV vector with an AAV-permissive cell for a time sufficient for the AAV-permissive cell to express the enzyme; b) lysing the cell and exposing the expressed enzyme, in a reaction medium, to a cleavable substrate specific for the enzyme; and c) assessing, by way of chromatography, a quantity or concentration of the substrate or of a product resulting from cleavage of the substrate by the enzyme in the reaction medium, wherein a finding that (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate identifies the AAV vector as being efficacious for treating the disease.

In another aspect, the disclosure provides a method of producing an AAV vector encoding an enzyme for treatment of a loss-of-function disease associated with a deleterious mutation in a gene encoding the enzyme in a patient in need thereof, the method including: a) contacting the AAV vector with an AAV-permissive cell expressing a cleavable substrate specific for the enzyme for a time sufficient for the AAV-permissive cell to express the enzyme; b) assessing, by way of chromatography, a quantity or concentration of the substrate or of a product resulting from cleavage of the substrate by the enzyme in the cell or surrounding extracellular media; and c) releasing the AAV vector for treating the disease if (i) the quantity or concentration of the cleavage product in the cell or surrounding extracellular media is increased relative to a reference quantity or concentration of the cleavage product or (ii) the quantity or concentration of the substrate in the cell or surrounding extracellular media is decreased relative to a reference quantity or concentration of the substrate.

In a further aspect, the disclosure provides a method of producing an AAV vector encoding an enzyme for treatment of a loss-of-function disease associated with a deleterious mutation in a gene encoding the enzyme in a patient in need thereof, the method including: a) contacting the AAV vector with an AAV-permissive cell for a time sufficient for the AAV-permissive cell to express the enzyme; b) lysing the cell and exposing the expressed enzyme, in a reaction medium, to a cleavable substrate specific for the enzyme; c) assessing, by way of chromatography, a quantity or concentration of the substrate or of a product resulting from cleavage of the substrate by the enzyme in the reaction medium; and d) releasing the AAV vector for treating the disease if (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate.

In another aspect, the disclosure provides a method of producing an AAV vector encoding an enzyme for treatment of a loss-of-function disease associated with a deleterious mutation in a gene encoding the enzyme in a patient in need thereof, the method including: a) contacting the AAV vector with an AAV-permissive cell expressing a cleavable substrate specific for the enzyme for a time sufficient for the AAV-permissive cell to express the enzyme; b) determining, by way of chromatography, that (i) the quantity or concentration of the cleavage product in the cell or surrounding extracellular media is increased relative to a reference quantity or concentration of the cleavage product or (ii) the quantity or concentration of the substrate in the cell or surrounding extracellular media is decreased relative to a reference quantity or concentration of the substrate; and c) releasing the AAV vector for treating the disease.

In another aspect, the disclosure provides a method of producing an AAV vector encoding an enzyme for treatment of a loss-of-function disease associated with a deleterious mutation in a gene encoding the enzyme in a patient in need thereof, the method including: a) contacting the AAV vector with an AAV-permissive cell for a time sufficient for the AAV-permissive cell to express the enzyme; b) lysing the cell and exposing the expressed enzyme, in a reaction medium, to a cleavable substrate specific for the enzyme; c) determining, by way of chromatography, that (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate; and d) releasing the AAV vector for treating the disease.

In another aspect, the disclosure provides a method of treating a loss-of-function disease associated with a deleterious mutation in a gene encoding an enzyme in a patient in need thereof, the method including: a) contacting an AAV vector encoding the enzyme with an AAV-permissive cell expressing a cleavable substrate specific for the enzyme for a time sufficient for the AAV-permissive cell to express the enzyme; b) determining, by way of chromatography, that (i) the quantity or concentration of the cleavage product in the cell or surrounding extracellular media is increased relative to a reference quantity or concentration of the cleavage product or (ii) the quantity or concentration of the substrate in the cell or surrounding extracellular media is decreased relative to a reference quantity or concentration of the substrate; and c) administering a therapeutically effective amount of the AAV vector to the patient.

In another aspect, the disclosure provides a method of treating a loss-of-function disease associated with a deleterious mutation in a gene encoding an enzyme in a patient in need thereof, the method including: a) contacting the AAV vector with an AAV-permissive cell for a time sufficient for the AAV-permissive cell to express the enzyme; b) lysing the cell and exposing the expressed enzyme, in a reaction medium, to a cleavable substrate specific for the enzyme; c) determining, by way of chromatography, that (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate; and d) administering a therapeutically effective amount of the AAV vector to the patient.

In another aspect, the disclosure provides a method of treating a loss-of-function disease associated with a deleterious mutation in a gene encoding an enzyme in a patient in need thereof, the method comprising administering to the patient an AAV vector encoding the gene, wherein the AAV vector has been identified as efficacious for treating the disease by way of a method comprising: a) contacting the AAV vector with an AAV-permissive cell expressing a cleavable substrate specific for the enzyme for a time sufficient for the AAV-permissive cell to express the enzyme; and b) determining, by way of chromatography, that (i) a quantity or concentration of a product resulting from cleavage of the substrate by the enzyme in the cell or surrounding extracellular media is increased relative to a reference quantity or concentration of the cleavage product or (ii) a quantity or concentration of the substrate in the cell or surrounding extracellular media is decreased relative to a reference quantity or concentration of the substrate.

In another aspect, the disclosure provides a method of treating a loss-of-function disease associated with a deleterious mutation in a gene encoding an enzyme in a patient in need thereof, the method comprising administering to the patient an AAV vector encoding the gene, wherein the AAV vector has been identified as efficacious for treating the disease by way of a method comprising: a) contacting the AAV vector with an AAV-permissive cell for a time sufficient for the AAV-permissive cell to express the enzyme; b) lysing the cell and exposing the expressed enzyme, in a reaction medium, to a cleavable substrate specific for the enzyme; and c) determining, by way of chromatography, that (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate.

In some embodiments of any of the foregoing aspects, the expressed enzyme is not purified from the cell lysate resulting from the lysis step prior to exposing the enzyme to the cleavable substrate.

In some embodiments of any of the foregoing aspects, the enzyme is a phosphatase. For example, in some embodiments, the phosphatase is a lipid phosphatase (e.g., MTM1, Myotubularin-related protein 1 (MTMR1), MTMR2, MTMR3, MTMR4, MTMR5, MTMR6, MTMR7, MTMR8, MTMR9, MTMR10, MTMR11, MTMR12, MTMR12, MTMR13, MTMR14, synaptojanin 1, or phosphatase and tensin homolog deleted on chromosome 10). In some embodiments, the lipid phosphatase is MTM1.

In some embodiments of any of the foregoing aspects, the determining of a quantity or concentration of a product resulting from cleavage of the substrate by the enzyme further includes determining a quantity or concentration of a marker. For example, in some embodiments, the marker is a fluorescently labelled lipid (e.g., a fluorescently labelled phospholipid), molecule, or protein. In some embodiments, the fluorescently labelled lipid is a fluorescently labelled phospholipid (e.g., phosphatidylinositol 3-phosphate). In some embodiments, the fluorescently labelled phospholipid is phosphatidylinositol 3-phosphate. In some embodiments the marker is a genetically encoded florescent marker (e.g., green fluorescent protein, yellow fluorescent protein, blue fluorescent protein, cyan fluorescent protein, red fluorescent protein, mCherry, dsRed, luciferase, β-galactosidase, or chloramphenicol acetyltransferase).

In some embodiments of any of the foregoing aspects, the AAV-permissive cell is a C2C12 cell, a Huh7 cell, a Hela cell, a HelaS3 cell, a Hepa1-6 cell, a HEK293 cell, a HepG2 cell, or an IMY-N9 cell. For example, in some embodiments, the AAV-permissive cell is a C2C12 cell. In some embodiments, the AAV-permissive cell (e.g., a C2C12 cell) is lysed.

In some embodiments of any of the foregoing aspects, the chromatography is high-performance liquid chromatography (HPLC), thin layer chromatography, column chromatography, ion-exchange chromatography, gel permeation chromatography, affinity chromatography, paper chromatography, gas chromatography, hydrophobic interaction chromatography, or pseudo affinity chromatography. For example, in some embodiments, the chromatography is HPLC.

In some embodiments of any of the foregoing aspects, the HPLC is normal phase HPLC, reverse-phase HPLC, hydrophobic interaction chromatography, or ion chromatography. For example, in some embodiments, the HPLC is normal phase HPLC.

In some embodiments of any of the foregoing aspects, the reference quantity or concentration is the expression level of the cleavage product in the AAV-permissive cell prior to contacting the AAV-permissive cell with the AAV vector.

In some embodiments of any of the foregoing aspects, the reference quantity or concentration is the level of the cleavage product or substrate in the reaction medium prior to exposing the expressed enzyme to the substrate.

In some embodiments of any of the foregoing aspects, the AAV vector includes capsid proteins from an AAV serotype selected from the group consisting of AAV1, AAV2, AAV3, AAV4, AAV5, AAV6, AAV7, AAV8, AAV9, AAVrh10, and AAVrh74.

In some embodiments of any of the foregoing aspects, the AAV vector is a pseudotyped AAV. In some embodiments, the pseudotyped AAV is AAV2/9. In some embodiments, the pseudotyped AAV is AAV2/8.

In some embodiments of any of the foregoing aspects, the AAV vector includes a recombinant capsid protein.

In some embodiments of any of the foregoing aspects, the enzyme is MTM1.

In some embodiments of any of the foregoing aspects, the MTM1 has a nucleic acid sequence that is at least 85% (e.g., at least 86%, 87%, 88%, 89%, 90% 91%, 92%, 93%, 94%, 95%, 96%, 97%, 98%, or 99%) identical to the nucleic acid of sequence of SEQ ID NO: 3. In some embodiments, the MTM1 has the nucleic acid of SEQ ID NO: 3.

In some embodiments of any of the foregoing aspects, the AAV vector further includes a muscle specific promoter (e.g., a desmin promoter, a phosphoglycerate kinase promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3). For example, in some embodiments, the muscle specific promoter is a desmin promoter. In some embodiments, the desmin promoter is a human desmin promoter. In some embodiments, the promoter is positioned 5′ to a polynucleotide encoding the enzyme.

In some embodiments of the foregoing aspect, the desmin promoter has a nucleic acid sequence that is at least 85% (e.g., at least 86%, 87%, 88%, 89%, 90% 91%, 92%, 93%, 94%, 95%, 96%, 97%, 98%, or 99%) identical to the nucleic acid of sequence of SEQ ID NO: 1. In some embodiments, the desmin promoter has the nucleic acid of SEQ ID NO: 1.

In some embodiments of any of the foregoing aspects, the AAV vector further includes a polyadenylation site (pA e.g., a simian virus 40 (SV40) late polyadenylation site, a SV40 early polyadenylation site, a human β-globin polyadenylation site, or a bovine growth hormone polyadenylation site). For example, in some embodiments, the pA site includes the SV40 late polyadenylation site. In some embodiments, the pA is positioned 3′ to a polynucleotide encoding the enzyme.

In some embodiments of the foregoing aspect, the AAV vector further includes an intron (e.g., an SV40 intron). For example, in some embodiments, the intron is an SV40 intron. In some embodiments, the intron is positioned 3′ to the promoter and 5′ to a polynucleotide encoding the enzyme.

In some embodiments of any of the foregoing aspects, the AAV vector has a nucleic acid sequence that is at least 85% (e.g., at least 86%, 87%, 88%, 89%, 90% 91%, 92%, 93%, 94%, 95%, 96%, 97%, 98%, or 99%) identical to the nucleic acid sequence of SEQ ID NO: 2. In some embodiments, the AAV vector has the nucleic acid of SEQ ID NO: 2.

In some embodiments of any of the foregoing aspects, the AAV vector is resamirigene bilparvovec.

In some embodiments of the foregoing aspects, the patient in need thereof is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the AAV vector. For example, in some embodiments, the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the AAV vector.

In some embodiments of the foregoing aspects, the patient requires mechanical ventilatory support. For example, in some embodiments, mechanical ventilatory support includes invasive mechanical ventilatory support and noninvasive mechanical ventilatory support.

In some embodiments of the foregoing aspects, the AAV vector is administered to the patient in an amount of from about 3×10¹³ vg/kg to about 2.3×10¹⁴ vg/kg (e.g., 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg). For example, in some embodiments, the AAV vector is administered to the patient in an amount of about 1.3×10¹⁴ vg/kg.

In some embodiments of the foregoing aspects, the AAV vector is administered to the patient in a single dose including the amount. In some embodiments of the foregoing aspects, the AAV vector is administered to the patient in two or more (e.g., two or more, three or more, four or more, five or more, six or more, seven or more, eight or more, nine or more, or ten or more) doses that, together, include the amount. In some embodiments, the AAV vector is administered to the patient in two or more doses (e.g., two or more, three or more, four or more, five or more, six or more, seven or more, eight or more, nine or more, or ten or more) that each, individually, include the amount.

In some embodiments of the foregoing aspects, the two or more doses are separated from one another by one year or more. In some embodiments, the two or more doses are administered to the patient within about 12 months (e.g., about 11 months, about 10 months, about 9 months, about 8 months, about 7 months, about 6 months, about 5 months, about 4 months, about 3 months, about 2 months, or about 1 month) of one another.

In some embodiments of the foregoing aspects, the AAV vector is administered to the patient by way of intravenous, intramuscular, intradermal, or subcutaneous administration.

In some embodiments of the foregoing aspects, upon administering the AAV vector to the patient, the patient displays a change from baseline in quantitative analysis of enzyme (e.g., MTM1) expression in a muscle biopsy. For example, in some embodiments, the patient displays the change from baseline in quantitative analysis of enzyme expression in a muscle biopsy by about 24 weeks after administration of the AAV vector to the patient.

In some embodiments of any of the foregoing aspects, the loss-of-function disease is a congenital loss-of-function disease (e.g., X-linked myotubular myopathy (XLM™)). For example, in some embodiments, the congenital loss-of-function disease is XLM™.

In another aspect, the disclosure provides a kit including an AAV vector encoding an enzyme and a package insert, wherein the package insert instructs a user of the kit to determine the efficacy of the AAV vector in accordance with the method of any one of the foregoing aspects.

### BRIEF DESCRIPTION OF THE DRAWINGS

FIG. 1 is a schematic illustrating the cleavage of phosphate groups (circled) from labelled (e.g., BODIPY fluorescently labelled (“FL”)) phosphatidylinositol 3-phosphate (PI (3) P), by the phosphatase Myotubularin 1 (MTM1), resulting in FL phosphatidylinositol (PI; FL-PI).

FIG. 2 is a chromatogram (e.g., a high-performance liquid chromatogram) of chloroform/methanol-extracted PI (3) P and PI on a column.

FIGS. 3A-3B are a schematic and a graph, respectively, of an experiment in which the amount of PI (ng) varied as a function of the titer of an adeno-associated virus (AAV) encoding MTM1 transfected into cells (e.g., C2C12 cells). From right-to-left, FIG. 3A is an experimental schematic depicting an AAV encoding the phosphatase MTM1 transfected into AAV-permissive C2C12 cells for a time sufficient for the AAV-permissive cell to express the MTM1, lysing the cells, and contacting them with FL-PI (3) P, as described in FIG. 1, to enable the cleavage of FL-PI (3) P to FL-PI by MTM1. FL-PI (3) P or FL-PI is then detected by high-performance liquid chromatography (HPLC) and quantified. FIG. 3B is a quantification of the concentration of FL-PI, as described in FIG. 3A, as a function of the titer (e.g., 5×10¹¹ (“E.00E+11”)) and payload (“AAV-DES-MTM1” encodes MTM1, while “AAV-MCK-GAA” encodes acid alpha-glucosidase) of the AAV that was transfected, respectively.

FIG. 4 is a quantification of the concentration of PI (3) P, as described in FIG. 3A, as a function of the titer (e.g., E.00E+11) and payload of the AAV that was transfected, respectively, as described in FIG. 3B.

### DEFINITIONS

As used herein, the term “about” refers to a value that is within 10% above or below the value being described. For example, “100 pounds” as used in the context of weight described herein includes quantities that are within 10% above or below 100 lbs. Additionally, when used in the context of a list of numerical quantities, it is to be understood that the term “about,” when preceding a list of numerical quantities, applies to each individual quantity recited in the list.

The term “AAV-permissive cell” refers to one or more cells in which an adeno-associated virus (AAV) can efficiently transfer genes. For example, such cells may include cells originating from the muscle, brain, and/or liver. Exemplary AAV-permissive cells include, but are not limited to, C2C12 cells, Huh7 cells, Hela cells, HelaS3 cells, Hepa1-6 cells, HEK293 cells, HepG2 cells, and IMY-N9 cells.

As used herein, the term “adeno-associated virus” (AAV), includes but is not limited to AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., Fields et al. Virology, 4th ed. Lippincott-Raven Publishers, Philadelphia, 1996. Additional AAV serotypes and clades have been identified recently. (See, e.g., Gao et al. J. Virol. 78:6381 (2004); Moris et al. Virol.33:375 (2004). The genomic sequences of various serotypes of AAV, as well as the sequences of the native ITRs, Rep proteins, and capsid subunits are known in the art. Such sequences may be found in the literature or in public databases such as GenBank. See, e.g., GenBank Accession Numbers NC-002077, NC-001401, NC-001729, NC-001863, NC-001829, NC-001862, NC-000883, NC-001701, NC-001510, NC-006152, NC-006261, AF063497, U89790, AF043303, AF028705, AF028704, J02275, J01901, J02275, X01457, AF288061, AH009962, AY028226, AY028223, AY631966, AX753250, EU285562, NC-001358, NC-001540, AF513851, AF513852 and AY530579; the disclosures of which are incorporated by reference herein for teaching AAV nucleic acid and amino acid sequences. See also, e.g., Bantel-Schaal et al. J. Virol. 73:939 (1999); Chiorini et al. J. Virol.71:6823 (1997); Chiorini et al. J. Virol.73:1309 (1999); Gao et al. Proc. Nat. Acad. Sci. USA 99:11854 (2002); Moris et al. Virol.33:375 (2004); Muramatsu et al. Virol.221:208 (1996); Ruffing et al. J. Gen. Virol.75:3385 (1994); Rutledge et al. J. Virol.72:309 (1998); Schmidt et al. J. Virol.82:8911 (2008); Shade et al. J. Virol. 58:921 (1986); Srivastava et al. J. Virol.45:555 (1983); Xiao et al. J. Virol.73:3994 (1999); WO 00/28061, WO 99/61601, WO 98/11244; and U.S. Pat. No. 6,156,303; the disclosures of which are incorporated by reference herein for teaching AAV nucleic acid and amino acid sequences.

As used herein in the context of an enzyme of interest, such as Myotubularin 1 (MTM1), the term “activity” refers to the biological functionality that is associated with a wild-type form of the enzyme. For example, the term “activity” refers to the ability of the enzyme to effectuate substrate turnover in a manner that yields the cleavage product of a corresponding chemical reaction. Activity levels of enzymes, such as MTM1, can be detected and quantitated, for example, using the methods described herein as well as substrate turnover assays known in the art.

As used herein, the terms “administering,” “administration,” and the like refer to directly giving a patient a therapeutic agent (e.g., a pharmaceutical composition including an AAV encoding an enzyme) by any effective route. Exemplary routes of administration are described herein and include systemic administration routes, such as intravenous injection, as well as routes of administration directly to the central nervous system of the patient, such as by way of intrathecal injection or intracerebroventricular injection, among others.

As used herein, the term “chromatography” refers to a technique for the separation of a mixture by passing the mixture in solution or suspension or as a vapor through a medium in which the components move at different rates. Exemplary types of chromatography include, but are not limited to, high-performance liquid chromatography, thin layer chromatography, column chromatography, ion-exchange chromatography, gel permeation chromatography, affinity chromatography, paper chromatography, gas chromatography, hydrophobic interaction chromatography, or pseudo affinity chromatography.

As used herein, the terms “effective amount,” “therapeutically effective amount,” and the like, when used in reference to a therapeutic composition, such as an AAV vector described herein, refer to a quantity sufficient to, when administered to the subject, including a mammal, for example a human, effect beneficial or desired results, such as clinical results. For example, in the context of treating a loss-of-function disease (e.g., a congential loss-of-function disease), such as XLM™, these terms refer to an amount of the composition sufficient to achieve a treatment response as compared to the response obtained without administration of the composition of interest. An “effective amount,” “therapeutically effective amount,” and the like, of a composition, such as an AAV vector of the present disclosure, also include an amount that results in a beneficial or desired result in a subject as compared to a control.

As used herein, the terms “encode” and “encoding” in the context of a gene (e.g., a gene encoding an enzyme) refer to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “extracellular media” refers to the endogenous collection of collagens, elastins, laminins, glycosaminoglycans, proteoglycans, antimicrobials, chemoattractants, cytokines, growth factors, and other molecules located exterior to the cell membrane. As used herein, extracellular media also refers to the extracellular contents surrounding a cell after lysis.

As used herein, the terms “high-performance liquid chromatography” and “HPLC” are used interchangably and refer to a technique in analytical chemistry used to separate, identify, and quantify each component in a mixture, as will be known to one of skill in the art. Exemplary types of HPLC include, but are not limited to, normal phase HPLC, reverse-phase HPLC, hydrophobic interaction chromatography HPLC, and ion chromatography.

The term “marker” refers to a chemical moiety useful as a label, indicator, or contrast agent. A marker may be capable of being detected by liquid chromatagrphy (e.g., high-performance liquid chromatagrphy), absorption spectroscopy, luminescence spectroscopy, fluorescence spectroscopy, magnetic resonance spectroscopy (e.g., MRI), or radioisotope detection. The terms “florescent marker” and “fluorescent label” refer interchangably to a marker that can absorb electromagnetic energy and is capable of at least partially re-emitting some fraction of that energy as electromagnetic radiation. Suitable fluorescent moieties include, but are not limited to, Bodipy™ FL, coumarins and related dyes, xanthene dyes such as fluoresceins, rhodols, and rhodamines, resorufins, cyanine dyes, bimanes, acridines, isoindoles, dansyl dyes, aminophthalic hydrazides such as luminol, and isoluminol derivatives, aminophthalimides, aminonaphthalimides, aminoenzofurans, aminoquinolines, dicyanohydroquinones, semiconductor fluorescent nanocrystals, fluorescent proteins, and fluorescent europium and terbium complexes and related compounds. In some embodiments, a marker can be a member of a specific binding pair, or can be associated (e.g., covalently) with a member of a specific binding pair. Specific binding pairs are pairs of molecules that are capable of specific interaction with one another, e.g., have an affinity for one another. For example, a specific binding pair can be ligand-protein binding pairs, e.g., enzyme-substrate, biotin-streptavidin, or epitope-antibody binding pairs. A binding pair that includes a marker has a larger apparent size than a corresponding binding pair that does not include a marker, and a larger apparent size than either member of the binding pair alone. Complexes of binding pairs can be detected by a method described herein or by other methods known to those of skill in the art, e.g., a chromatographic assay, an immunoassay format, or a gel shift assay.

As used herein, the term “loss-of-function disease” refers to a disease caused by a loss-of-function mutation, which is a type of mutation in which the alterned gene product lacks the moleculer function of the wild-type gene. For example, congenital loss-of-function diseases may include, but are not limited to, X-linked myotubular myopathy (XLM™). In some embodiments, a loss-of-function disease may be congenital.

As used herein, the term “sample” refers to a specimen isolated from a cell or the surrounding extracellular media thereof. The cell may be, for example, a cell that was contacted with an AAV vector encoding an enzyme (e.g., MTM1).

As used herein, the term “substrate” refers to a molecule upon which an enzyme (e.g. Myotublarin 1) acts (e.g., hydrolizes). For example, a “non-phosphorylated substrate” is distinguished from a “phosphorylated substrate” primarily by the presence of a phosphate group that has or has not, respectively, been hydrolyzed by a phosphatase.

As used herein, the term “level” refers to a level of a molecule or protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a molecule or protein may be expressed in mass/vol (e.g., g/dL, mg/mL, ug/mL, or ng/ml) or percentage relative to total molecules or protein, respectively, in a sample.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, the terms “patient” and “subject” refer to an organism that receives treatment for a particular disease or condition as described herein (such as a loss-of-function disease, e.g., XLM™). Examples of subjects and patients include mammals, such as humans, receiving treatment for a disease or condition described herein.

By a “reference” is meant any useful reference used to compare cleavage product levels in a sample that contains a substrate that is cleaved by an enzyme (e.g., an enzyme that is encoded by a vector). The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same sample (e.g., a cell or tissue) that has not been exposed to the enzyme; a sample that has been contacted with an enzyme that does not cleave the substrate; or a sample of a purified substrate (e.g., any described herein) at a known normal concentration. By “reference standard or level” is meant a value or number derived from a reference sample. A standard curve of levels of a purified substrate, e.g., any described herein, within the normal reference range can also be used as a reference. As described herein, the reference may be a quantity or a concentration.

In some embodiments, the reference quantity or concentration is the expression level of an enzyme cleavage product in an AAV-permissive cell prior to contacting the AAV-permissive cell with an AAV vector.

As used herein, the terms “treat” and “treatment” refer to therapeutic treatment, in which the object is to prevent or slow down (lessen) an undesired physiological change or disorder, such as the progression of a loss-of-function disease (e.g., a congenital loss-of-function disease), such as XLM™, among others. Beneficial or desired clinical results include, but are not limited to, alleviation of symptoms (e.g., stiffness and/or joint contractures), diminishment of extent of disease, stabilized (e.g., not worsening) state of disease, delay or slowing of disease progression, amelioration or palliation of the disease state, and remission (whether partial or total), whether detectable or undetectable. In the context of a loss-of-function disease (e.g., a congenital loss-of-function disease), such as XLM™, treatment of a patient may manifest in one or more detectable changes, such as an increase in the concentration of MTM1 protein or nucleic acids (e.g., DNA or RNA, such as mRNA) encoding MTM1, or an increase in MTM1 activity (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or more. The concentration of MTM1 protein may be determined using protein detection assays known in the art, including ELISA assays described herein. The concentration of MTM1-encoding nucleic acids may be determined using nucleic acid detection assays (e.g., RNA Seq assays) described herein. Additionally, treatment of a patient suffering from a loss-of-function disease (e.g., a congenital loss-of-function disease), such as XLM™, may manifest in improvements in a patient's muscle function (e.g., skeletal muscle function) as well as improvements in muscle coordination. For example, manifestation of an improvement may include increasing diaphragm and/or respiratory muscle progression.

As used herein, the terms “X-linked myotubular myopathy” and “XLM™” refer to the congenital loss-of-function disease that is caused by mutations of the MTM1 gene and is characterized by symptoms including mild to profound muscle weakness, hypotonia (diminished muscle tone), feeding difficulties, and/or severe breathing complications. Human MTM1 has NCBI Gene ID NO 4534. An exemplary wild-type human MTM1 nucleic acid sequence is provided in NCBI RefSeq Acc. No. NM_000252.3, and an exemplary wild-type myotubularin 1 amino acid sequence is provided in NCBI RefSeq Acc. No. NP_000243.1 (SEQ ID NO: 4).

### DETAILED DESCRIPTION

The present disclosure provides methods that can be used for determining the efficacy of an adeno-associated virus (AAV) vector encoding an enzyme, for example for treatment of a loss-of-function disease (e.g., a congenital loss-of-function disease e.g., X-linked myotubular myopathy). In accordance with the methods and uses described herein, a patient (e.g., a human patient) having a loss-of-function disease (e.g., a congenital loss-of-function disease e.g., XLM™) may be administered an AAV that encodes an enzyme (e.g., Myotubularin 1 (MTM1)). The method may include, for example, contacting the AAV vector with an AAV-permissive cell expressing a cleavable substrate specific for the enzyme for a time sufficient for the AAV-permissive cell to express the enzyme and assessing, then, by way of high-performance liquid chromatography (HPLC), a quantity or concentration of a product resulting from cleavage of the substrate by the enzyme in the cell may be assessed or determined. Alternatively, the method may include contacting the AAV vector with an AAV-permissive cell for a time sufficient for the AAV-permissive cell to express the enzyme, lysing the cell and exposing the expressed enzyme, in a reaction medium, to a cleavable substrate specific for the enzyme, and then, by way of HPLC, a quantity or concentration of a product resulting from cleavage of the substrate by the enzyme in the reaction medium may be assessed or determined. In some embodiments, a finding that the quantity or concentration of the cleavage product in the cell, surrounding extracellular media, or reaction medium is increased relative to a reference quantity or concentration of the cleavage product identifies the AAV vector as being efficacious for treating the disease. In some embodiments, a finding that the quantity or concentration of the substrate in the cell, surrounding extracellular media, or reaction medium is decreased relative to a reference quantity or concentration of the substrate identifies the AAV vector as being efficacious for treating the disease. An exemplary AAV vector that may be used in conjunction with the methods and uses of the disclosure is resamirigene bilparvovec.

The present disclosure is based, at least in part, on the discovery of methods that are surprisingly useful in determining the efficacy of an AAV, for example, in a batch release assay. The disclosed methods, therefore, address a significant need associated with existing methods of AAV potency assays, such as the outstanding need for high-throughput, quantitative measurements of potency. For example, whereas existing AAV potency assays using thin-layer chromatography enable a qualitative analysis of the number of components in a mixture, the HPLC methods described herein enable both high detection sensitivity and quantification, which is an essential output measure to determine potency of an AAV payload. Surprisingly, the detection and quantification of product resulting from a cleavable substrate specific to the expressed enzyme was successfully performed directly on cell lysate samples. For example, the detection and quantification of product resulting from a cleavable substrate specific to the expressed enzyme was successfully performed using cell lysate samples without purifying the expressed enzyme prior to the exposure of the enzyme to the cleavable substrate. An aspect of the applicant's teachings is to provide a method for an AAV potency assay, including: contacting an AAV-permissive cell with an AAV encoding an enzyme for a time sufficient for the AAV-permissive cell to express the enzyme; lysing the cells; incubating the cells or the expressed enzyme with a phosphorylated substrate labeled with an marker; and assessing, by way of HPLC, the quantity or concentration of the labelled cleavage product as compared to the quantity or concentration of the phosphorylated substrate labeled with a marker (e.g., uncleaved).

In some embodiments, the disclosure describes a method of producing an AAV vector encoding an enzyme for treatment of a loss-of-function disease associated with a deleterious mutation in a gene encoding the enzyme in a patient, including determining the efficacy of an AAV vector encoding an enzyme (e.g., MTM1) by assessing a quantity or concentration of a product resulting from cleavage of a substrate by the enzyme by way of HPLC in an AAV-permissive cell, surrounding extracellular media, or a reaction medium.

In some embodiments, the disclosure describes a method of treating a loss-of-function disease associated with a deleterious mutation in a gene encoding an enzyme in a patient, including determining the efficacy of an AAV vector encoding an enzyme (e.g., MTM1) by assessing a quantity or concentration of a product resulting from cleavage of a substrate by the enzyme by way of HPLC in an AAV-permissive cell, surrounding extracellular media, or a reaction medium; and administering a therapeutically effective amount of the AAV vector to the patient.

The sections that follow provide a description of therapeutic enzymes (e.g., phosphatases), exemplary loss-of-function diseases associated with a deleterious mutation in a gene encoding an enzyme, exemplary AAVs, and methods of HPLC for assessing a quantity or concentration of a product resulting from cleavage of a substrate by the enzyme described herein. The following sections also describe various transduction agents, pharmaceutical compositions, and routes of administration that may be used in conjunction with the compositions and methods of the disclosure.

### Therapeutic Enzymes

Enzymes that can be encoded by an AAV and whose respective cleavage product can be assessed according to the methods described herein included therapeutic enzymes such as those that can be transferred to a subject (e.g., a human patient) suffering from a disease or condition characterized by a deficiency in the enzyme. For instance, enzymes that can be encoded by an AAV described herein include myotubularin 1 (MTM1), myotubularin-related protein 1 (MTMR1), MTMR2, MTMR3, MTMR4, MTMR5, MTMR6, MTMR7, MTMR8, MTMR9, MTMR10, MTMR11, MTMR12, MTMR12, MTMR13, MTMR14, synaptojanin 1, phosphatase and tensin homolog deleted on chromosome 10, acid α-glucosidase (GAA), carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, porphobilinogen deaminase, factor VII, factor IX, thrombin, factor X, factor XI, superoxide dismutase, glutathione peroxidase and reductase, heme oxygenase, angiotensin converting enzyme, urokinase, plasminogen activator, cystathione beta-synthase, branched chain ketoacid decarboxylase, isovaleryl-CoA dehydrogenase, propionyl CoA carboxylase, methyl malonyl CoA mutase, glutaryl CoA dehydrogenase, beta-glucosidase, pyruvate carboxylase, hepatic phosphorylase, phosphorylase kinase, glycine decarboxylase (also referred to as P-protein), Cu/Zn superoxide dismutase, aromatic amino acid decarboxylase, tyrosine hydroxylase, acetylcholine synthetase, prohormone convertases, lactase, lipase, trypsin, gastrointestinal enzymes including chymotrypsin, and pepsin, adenosine deaminase, al anti-trypsin, tissue inhibitor of metalloproteinases, trehalose phosphate synthase, hexokinases I, II and III, glucokinase, and suicide genes such as thymidine kinase and cytosine deaminase. Other useful enzymes include those involved in lysosomal storage disorders, including acid β-glucosidase, α-galactosidase a, α-1-iduronidase, iduroate sulfatase, lysosomal acid α-glucosidase, sphingomyelinase, hexosaminidase A, hexomimidases A and B, arylsulfatase A, acid lipase, acid ceramidase, galactosylceramidase, α-fucosidase, aspartylglucosaminidase, neuramidase, galactosylceramidase, heparan-N-sulfatase, N-acetyl-α-glucosaminidase, Acetyl-CoA: α-glucosaminide N-acetyltransferase, N-acetylglucosamine-6-sulfate sulfatase, N-acetylgalactosamine-6-sulfate sulfatase, arylsulfatase B, β-glucuoronidase and hexosaminidases A and B. In some embodiments, the enzyme is MTM1. In some embodiments, the enzyme is GAA.

Representative enzymes associated with a disease or condition characterized by a deficiency in the enzyme include those listed in Table 1, below.

TABLE 1
Exemplary disorders associated with an enzyme deficiency

| | Disease associated with | Exemplary wild-type |
|---|---|---|
| | deficiency in enzyme or | or variant |
| Enzyme of component thereof | component thereof | enzyme sequence |
| Myotubularin 1 (MTM1) | X-linked myotubular myopathy | NP_000243.1 |
| acid α-glucocidase (GAA) | Pompe | NP_000143.2, |
| | | NP_001073271.1, |
| | | NP_001073272.1 |
| Aromatic L-amino acid | Parkinson's disease | |
| docarboxylase (AADC) | | NP_00781.1, |
| | | NP_001076440.1, |
| | | NP_001229815.1, |
| | | NP_001229816.1, |
| | | NP_001229817.1, |
| | | NP_001229818.1, |
| | | NP_001229819.1 |
| Glutamate decarboxylase 1 | Parkinson's disease | NP_00808.2, NP_038473.2 |
| (GAD1) | | |
| Glutamate decarboxylase 2 | Parkinson's disease | NP_000809.1, |
| (GAD2) | | NP_001127838.1 |
| Asparto [symbol] cylase (ASPA) | Canavan's disease | NP_000040.1, |
| | | NP_001121557.1 |
| ATPase, Ca² [symbol] transporting, | Chronic heart failure | NP_001672.1, |
| cardiac muscle, slow twitch 2 | | NP_733765.1 |
| (SERCA2) | | |
| M-acetylglucosaminidase, α | Sanfilippo syndrome (MPSIIB) | NP_000254.2 |
| (NAGLU) | | |
| Iduronidase, α-1 (IDUA) | MPSI-Hurler | NP_000194.2 |
| Iduronate 2-sulfatase (IDS) | MPSII-Hunter | NP_000193.1, |
| | | NP_001160022.1, |
| | | NP_006114.1 |
| Glucuronidase [symbol] β (GUSB) | MPSVII-Sly | NP_000172.2, NP 001271219.1 |
| Hexosaminidase A, α | Tay-Sachs | NP_000511.2 |
| polypeptide (HEXA) | | |
| ApaL [symbol] | mitochondrial heteroplasmy, | YP_007161330.1 |
| | my [symbol] lonic epilepsy with ragged | |
| | red fibers (MERRF) or | |
| | mitochondrial | |
| | encephalomyopathy, | |
| | lactic acidosis, and stroke-like | |
| | episodes (ME LAS) | |
| NADH ubiquinone | Leber hereditary optic | YP_003024035.1 |
| oxidoreductase subunit 4 (ND4) | | |
| very long-acyl-CoA | very long-chain acyl-CoA | NP_000009.1, |
| dehydrogenase (VLCAD) | dehydrogenase (VLCAD) | NP_001029031.1, |
| | deficiency | NP_001257376.1, |
| | | NP_001257377.1 |
| short-chain acyl-CoA | short-chain acyl-CoA | NP_000008.1 |
| dehydrogenase (SCAD) | dehydrogenase (SCAD) | |
| | deficiency | |
| medium-chain acyl-CoA | medium-chain acyl-CoA | NP_000007.1, |
| dehydrogenase (MCAD) | dehydrogenase (MCAD) | NP_001120800.1, |
| | deficiency | NP_001272971.1, |
| | | NP_001272972.1, |
| | | NP_001272973.1 |
| Myophosphorylase (PYGM) | McArdle disease (glycogen | NP_001158188.1, |
| | storage disease type V, | NP_005600.1 |
| | myophosphorylase deficiency) | |
| Lipoprotein lipase (LPL) | LPL deficiency | NP_000228.1 |
| Glucocerebrosidase (GC) | Gaucher disease | NP_000148.2, |
| | | NP_001005741.1, |
| | | NP_001005742.1, |
| | | NP_001165282.1, |
| | | NP_001165283.1 |
| UDP glucuronosyltransferase 1 | Crig [symbol] er-Naijar syndrome | NP_000454.1 |
| family member A1 (UGT1A1) | | |
| Glucose 6-phosphatase | GSD- [symbol] | NP_000142.2 [symbol] |
| (G6Pase) | | NP_001257326.1 |
| Omithine carbamoyltransferase | OTC deficiency | NP_000522.3 |
| (OTC) | | |
| Cystathionine-β-synthase (CBS) | Homocystinuria | NP_000062.1, |
| | | NP_001171479.1, |
| | | NP_001171480.1 |
| Galactosidase, α (AGA) | Fabry disease | NP_000160.1 |
| Phenylalanine hydroxylase | Hypercholesterolaemia or | NP_000268.1 |
| (PAH) | Phenylketonuria (PKU) | |
| Propionyl CoA carboxylase, | Propionic acida [symbol] mias | NP_000273.2, |
| alpha polypeptide (PCCA) | | NP_001121164.1, |
| | | NP_001171475.1 |

[symbol]

indicates data missing or illegible when filed

In some embodiments, it is assessed that there is an increase or a decrease in quantity or concentration of a cleavage product of an enzyme described herein relative to a reference quantity or concentration. In some embodiments, this finding of a changed quantity or concentration of a cleavage product of an enzyme relative to a reference quantity or concentration identifies an AAV described herein as being efficacious for treating a loss-of-function disease, as exemplified in Table 1 above.

In some embodiments, it is determined that there is an increase or a decrease in quantity or concentration of a cleavage product of an enzyme described herein relative to a reference quantity or concentration. In some embodiments, this determination identifies an AAV described herein as being efficacious for treating a loss-of-function disease, as exemplified in Table 1 above.

### Therapeutic Phosphatases

Phosphatases are a type of enzyme that dephosphorylate a substrate; e.g., a phosphatase hydrolyzes phosphoric acid monoesters into a phosphate ion and a molecule with a free hydroxyl group. Phosphatases can be categorized into two main categories: cysteine-dependent phosphatases (CDPs) and metallo-phosphatases. Metallo-phosphatases are dependent on the presence of one or more metal ions in their active site(s) for activity.

CDPs catalyze the hydrolysis of a phosphoester bond via a phospho-cysteine intermediate. The free cysteine nucleophile forms a bond with the phosphorus atom of the phosphate moiety, and the P—O bond linking the phosphate group to the tyrosine is protonated, either by a suitably positioned acidic amino acid residue or a water molecule. The phospho-cysteine intermediate is then hydrolyzed by another water molecule, thus regenerating the active site for another dephosphorylation reaction.

In contrast, metallo-phosphatases catalyze essential metal ion(s), such as magnesium, manganese, iron, zinc, or any combination thereof; within their active site. In this catalytic process, a hydroxyl ion bridging the two metal ions takes part in nucleophilic attack on the phosphate group.

Representative phosphatases whose respective cleavage product can be assessed according to the methods described herein include a lipid phosphatase, such as MTM1, MTMR1, MTMR2, MTMR3, MTMR4, MTMR5, MTMR6, MTMR7, MTMR8, MTMR9, MTMR10, MTMR11, MTMR12, MTMR12, MTMR13, MTMR14, synaptojanin 1, phosphatase and tensin homolog deleted on chromosome 10, and glucose-6-phosphatase. In some embodiments, the phosphatase is MTM1.

### An Exemplary Disorder Associated with a Phosphatase Deficiency: X-Linked Myotubular Myopathy

XLM™ is a rare, life-threatening, congenital myopathy caused by a loss-of-function mutation in the gene encoding the phosphatase MTM1 and is characterized in most patients by profound muscle weakness and hypotonia at birth, which results in severe respiratory insufficiency, inability to sit up, stand or walk, and early mortality.

The myopathy associated with XLM™ impairs the development of motor skills such as sitting, standing, and walking. Affected infants may also have difficulties with feeding due to muscle weakness. Individuals with this condition often do not have the muscle strength to breathe on their own and must be supported with mechanical ventilation. Some affected individuals require mechanical ventilation only periodically, such as during sleep, while others require mechanical ventilation continuously. Patients having XLM™ may also have weakness in the muscles that control eye movement (ophthalmoplegia), weakness in other muscles of the face, and absent reflexes (areflexia).

In XLM™, muscle weakness often disrupts normal bone development and can lead to fragile bones, an abnormal curvature of the spine (scoliosis), and joint deformities (contractures) of the hips and knees. Patients having XLM™ may have a large head with a narrow and elongated face and a high, arched roof of the mouth (palate). Patients may also have liver disease, recurrent ear and respiratory infections, or seizures.

As a consequence of their severe breathing difficulties, patients having XLM™ usually survive only into early childhood; however, some patients with this condition have lived into adulthood. The compositions and methods of the disclosure provide the important medical benefit of being able to prolong the lifetimes of such patients by restoring functional MTM1 expression.

### Detectable Marker

The methods and uses herein described include determining a quantity or concentration of a marker (e.g., a substrate of an enzyme described herein which is associated with a marker, such that the putative cleavage product is also associated with said marker).

The choice of a marker is governed by a number of factors including the mode of detection, the availability of specific instrumentation, and the ease of coupling of the marker to a substrate. Other factors that may be relevant to a particular use include the effect of a marker on the solubility of a substrate, the kinetics of the post-translational activity or protease activity with respect to a substrate, and the desired detection sensitivity of the AAV potency assay.

Numerous markers are commercially available or can be readily made. In general, a marker can exhibit an optical property, a magnetic property, or a radioactive property. Thus, once associated with a substrate, a marker allows a resulting substrate to exhibit an optical property, a magnetic property, or a radioactive property that is similar to or the same as that of the marker alone. In some embodiments, the association of a marker with a substrate may alter a detectable property of the marker to a greater or lesser extent. For example, conjugation of a fluorophore to a substrate may result in a composition having an emission maximum that is different from that of the fluorophore alone in solution. In other embodiments, a marker can be a member of a specific binding pair. For example, a marker can be the ligand member of a ligand-protein binding pair, e.g., the biotin member of the biotin-streptavidin binding pair.

For fluorescent markers, preferred fluorophores typically exhibit good quantum yields, long excited state lifetimes, and large extinction coefficients; are resistant to collisional quenching and bleaching; and should be easily conjugated to a peptide. Examples of illustrative fluorophores include cyanines, oxazines, thiazines, porphyrins, phthalocyanines, fluorescent infrared-emitting polynuclear aromatic hydrocarbons such as violanthrones, fluorescent proteins, near IR squaraine dyes.

Specific fluorophores include, without limitation, fluorescein isothiocyanate, 5-FAM (5-carboxyfluorescein), 6-FAM (6-carboxyfluorescein), 5,6-FAM, 7-hydroxycoumarin-3-carboxamide, 6-chloro-7-hydroxycoumarin-3-carboxamide, dichlorotriazinylaminofluorescein, tetramethylrhodamine-5 (and 6)-isothiocyanate, 1,3-bis-(2-diakylamino-5-thienyl)-substituted squarines, the succinimidyl esters of 5 (and 6) carboxyfluoroscein, 5 (and 6)-carboxytetramethylrhodamine, and 7-amino-4-methylcoumarin-3-acetic acid. Semiconductor fluorescent nanocrystals are available with a range of emission spectra, are highly fluorescent and are also useful (see Bruchez et aL, Science 281:2013-2016).

Genetically encoded fluorescent markers that may be used in the methods and uses of this disclosure include, but are not limited to, any one of the following green fluorescent protein (GFP), yellow fluorescent protein (YFP), blue fluorescent protein (BFP), cyan fluorescent protein (CFP), red fluorescent protein (RFP), mCherry, dsRed, luciferase (Luc), β-galactosidase (lacZ), chloramphenicol acetyltransferase (CAT), mTagBFP2, mTurquoise2, mCerulean3, EGFP, mWasabi, Superfolder GFP, mNeonGreen, mClover3, Venus, Citrine, mKOK, tdTomato, TagRFP-T, mRuby3, mScarlet, FusionRed, mCherry, mStable, mKate2, mMaroon1, mCardinal, T-Sapphire, mCyRFP1, LSSmOrange, or mBeRFP.

Lanthanide complexes (e.g., metal chelates of Eu or Tb) are also useful. Their long lifetimes may allow easy suppression of the autofluorescence of biological samples, as fluorescent signals may be measured after background signals have decayed. Accordingly, lanthanide complexes, such as Eu or Tb metal chelates, may be useful.

Illustrative luminescent moieties include chemiluminescent, electroluminescent, and bioluminescent compounds. Exemplary bioluminescent compounds include bioluminescent proteins such as firefly, bacterial, or click beetle luciferases, aequorins, and other photoproteins. Alternate electroluminescent moieties include ruthenium complexes, and alternate chemiluminescent moieties include those based on 1,2-dioxetanes.

Magnetic detectable moieties include magnetic resonance contrast agents, e.g., chelates of paramagnetic, ferromagnetic, or diamagnetic metal ions, or magnetic particles.

In some applications it may be desirable to derivatize a marker to render it more hydrophobic and permeable to cell membranes. The derivatizing groups may undergo hydrolysis inside cells to regenerate the compositions, thus trapping them within cells. For this purpose, it is preferred that phenolic hydroxyls or free amines in the structures are acylated with C1-C4 acyl groups (e.g., formyl acetyl, n-butyl) or converted to, e.g., esters and carbonates, as described in Bundgaard, H., Design of Prodrugs, Elsevier Science Publishers (1985), Chapter 1, page 3 et seq. Further modification of fluorescent moieties may also be accomplished e.g., as described in U.S. Pat. No. 5,741,657, incorporated herein by reference.

A marker may be attached to a substrate by a linker that provides a spacer between the marker and the substrate, thereby preventing steric or charge interference of the marker on the interaction between, e.g., the enzyme and the substrate. Preferred linkers are substantially stable under cellular conditions and easily coupled to a substrate and marker. Examples include flexible aliphatic linkers such as γ-amino-n-butyric acid (GABA), diaminopentane, and aminohexanoyl, as well as rigid aromatic linkers. Such linkers are known in the art and described for example in the Handbook of Fluorescent Probes and Research Chemicals, by Richard Hangland, published by Molecular Probes, incorporated herein by reference.

Non-covalent methods of attachment may also be used to associate a substrate with a marker. For example, a substrate may be designed to encompass a specific binding site for a marker, as described in pending U.S. Pat. Nos. 6,054,271; 6,008,378, and 5,932,474. Labeling may then be achieved by incubation of a substrate with a membrane-permeable fluorescent binding partner, which has the advantages of enabling the expression of substrates within intact living cells, and the subsequent labeling of these peptides in situ to create compositions of the presently disclosed subject matter within intact living cells (e.g., AAV-permissive cells).

In some embodiments the marker is a fluorescently labelled lipid. In some embodiments, the fluorescently labelled lipid is a fluorescently labelled phospholipid. In some embodiments, the fluorescently labelled phospholipid is phosphatidylinositol 3-phosphate.

### Chromatography

Chromatography is a technique for the separation of a mixture by passing it in solution or suspension or as a vapor through a medium in which the components move at different rates. Chromatography in the broadest sense refers to processes that permit the resolution of a mixture of components as a consequence of differences in the rates at which the individual components of that mixture migrate through what is referred to as a stationary phase or medium under the influence of a mobile phase.

Chromatography is used to separate mixtures of chemicals into individual components. Exemplary types of chromatography include, but are not limited to, high-performance liquid chromatography (HPLC), thin layer chromatography, column chromatography, ion-exchange chromatography, gel permeation chromatography, affinity chromatography, paper chromatography, gas chromatography, hydrophobic interaction chromatography, or pseudo affinity chromatography.

Ion exchange chromatography is a technique for separating molecules, typically ions or polar molecules, based on their ionic charge. Functionalized chromatography media for use in such methods therefore contain one or more moieties which are positively or negatively charged. Positive and/or negative charges in functionalized chromatography media are usually balanced with one or more counter ions. Ion exchange chromatography involves one or more of cation exchange chromatography and anion exchange chromatography.

Affinity chromatography is a technique for separating molecules based on their affinity to particular ligands, usually but not always biological ligands. This method may, for example, rely on the attractive forces between antibodies and antigens or enzymes and substrates. Functionalized chromatography media for use in affinity capture chromatography typically contain one or more moieties chosen from one or more proteins, peptides, antibodies or fragments thereof, dyes, histidine, or groups containing a metal cation. Alternatively, functionalized chromatography media for use in affinity chromatography may contain mimetic or synthetic ligands that mimic the action of protein ligands.

Paper chromatography is a method of separation in which a mobile phase passes through a filter paper material. The mixture, such as a line or spot of dye, is situated on the filter paper. When an end of the filter paper is exposed to an appropriate fluid mobile phase, such as a liquid solvent like water, capillary action causes the solvent to flow.

Hydrophobic interaction chromatography is a method for separating proteins based on the strength of their relative hydrophobic interactions with a hydrophobic adsorbent. Hydrophobicity is generally defined as the repulsion between a non-polar compound and a polar environment, such as water. Hydrophobic “interactions” are essentially the tendency of a polar environment to exclude non-polar (i.e., hydrophobic) compounds from the polar environment and force aggregation of the hydrophobic amongst themselves. The phenomenon of hydrophobic interactions is applied to the separation of proteins by using an aqueous salt solution to force a hydrophobic protein in a sample to aggregate with or bind adsorptively to hydrophobic functional groups (the adsorbent) affixed to a solid support. The adsorbed proteins are released from the adsorbent by eluting with decreasing salt concentrations which reverse the environment promoting the hydrophobic interactions, leading to loss of hydrophobic interactions between the proteins and the support and release of the protein from the support in order of increasing hydrophobicity (with the least hydrophobic proteins being released first).

In gel permeation chromatography, or size exclusion chromatography, the separation of components is a function of their molecular size and the stationary phase typically does not attract the components. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase. Size exclusion chromatography is widely utilized in a variety of scientific fields. In the biological sciences, size exclusion chromatography is used for the isolation and purification of biological molecules, such as peptides, hormones or DNA.

Thin layer chromatography is a technique in which small volumes of various solutions of the substance mixtures to be separated are applied to the porous thin layer in the form of spots on a starting line. The edge of the support plates below the starting line is then brought into contact with a liquid eluent, where the capillary rise of the eluent in the porous thin layer carries the individual substances along with it. Due to different adsorption or distribution coefficients, the substances are transported at different speeds and thus separated.

### High-Performance Liquid Chromatography

HPLC, formerly referred to as high-pressure liquid chromatography, is a technique in analytical chemistry used to separate, identify, and quantify each component in a mixture. In a conventional liquid chromatography system, a liquid solvent (referred to as the mobile phase) is introduced from a reservoir and is pumped through the liquid chromatography system. The mobile phase exits the pump under pressure. The mobile phase then travels via tubing to a sample injection valve. The sample injection valve allows an operator to inject a sample into the liquid chromatography system, where the sample will be carried along with the mobile phase.

Moreover, in a conventional LC system, the sample and mobile phase pass through one or more filters and often a guard column before coming to the column. In some embodiments, any suitable column may be used. In some embodiments the column is a chloroform/methanol. A typical column usually consists of a piece of steel tubing which has been packed with a packing material. The packing consists of the particulate material packed inside the column. It usually consists of silica-or polymer-based particles, which are often chemically bonded with a chemical functionality. The packing material is also known as the stationary phase. One of the fundamental principles of separation is the mobile phase continuously passing through the stationary phase. When the sample is carried through the column (along with the mobile phase), the various components (solutes) in the sample migrate through the packing within the column at different rates (e.g., there is differential migration of the solutes). Because of the different rates of movement, the components gradually separate as they move through the column. Differential migration is affected by factors such as the composition of the mobile phase, the composition of the stationary phase (e.g., the material with which the column is packed), and the temperature at which the separation takes place. Thus, such factors will influence the separation of the sample's various components. Once the sample (with its components now separated) leaves the column, it flows with the mobile phase past a detector. The detector detects the presence of specific molecules or compounds. Two general types of detectors are used in liquid chromatography applications. One type measures a change in some overall physical property of the mobile phase and the sample (such as their refractive index). The other type measures only some property of the sample (such as the absorption of ultraviolet radiation). A typical detector in a LC system can measure and provide an output in terms of mass per unit of volume (e.g., grams per milliliter) or mass per unit of time (e.g., grams per second) of the sample's components. From such an output signal, a chromatogram can be provided; the chromatogram can then be used by an operator to determine the chemical components present in the sample.

The difference between traditional liquid chromatography and HPLC is that the solvent in liquid chromatography travels by the force of gravity, such that HPLC relies on pumps to pass a pressurized liquid solvent containing the sample mixture through a column filled with a solid adsorbent material. column. HPLC techniques for use in the presently disclosed subject matter include, without limitation: normal phase HPLC, reverse-phase HPLC, hydrophobic interaction chromatography, and ion chromatography. In some embodiments, any suitable method of HPLC may be used in the methods described herein.

Normal-phase HPLC separates analytes based on their affinity for a polar stationary surface such as silica, hence it is based on analyte ability to engage in polar interactions (such as hydrogen-bonding or dipole-dipole type of interactions) with the sorbent surface. Normal-phase HPLC uses a non-polar, non-aqueous mobile phase (e.g., chloroform), and works effectively for separating analytes readily soluble in non-polar solvents. In contrast, reverse phase HPLC has a non-polar stationary phase and an aqueous, moderately polar mobile phase. In addition, other mobile phase modifiers can affect analyte retention. For example, the addition of inorganic salts causes a moderate linear increase in the surface tension of aqueous solutions, leading to an increase in retention time. This technique, called hydrophobic interaction chromatography, is used for mild separation and recovery of proteins and protection of their biological activity in protein analysis. Another method of HPLC, called ion chromatography, separates ions and polar molecules based on their affinity to the ion exchanger. The two types of ion chromatography are anion-exchange and cation-exchange. In either case, the charged molecules bind to moieties which are oppositely charged by forming ionic bonds to the insoluble stationary phase. The equilibrated stationary phase consists of an ionizable functional group where the targeted molecules of a mixture to be separated and quantified can bind while passing through the column. Cation-exchange chromatography is used when the molecule of interest is positively charged. In this type of chromatography, the stationary phase is negatively charged and positively charged molecules are loaded to be attracted to it. Anion-exchange chromatography is when the stationary phase is positively charged and negatively charged molecules are loaded to be attracted to it. The bound molecules then can be eluted and collected using an eluant which contains anions and cations by running higher concentration of ions through the column or changing pH of the column.

In some embodiments, the HPLC is normal phase HPLC. In some embodiment, the HPLC is reverse-phase HPLC. In some embodiments, the HPLC is hydrophobic interaction chromatography. In some embodiments, the HPLC is ion chromatography.

In some embodiments, it is assessed by HPLC that there is an increase in quantity or concentration of a cleavage product of an enzyme relative to a reference quantity or concentration. In some embodiments, it is assessed by HPLC that there is a decrease in quantity or concentration of a cleavage product of an enzyme relative to a reference quantity or concentration.

In some embodiments, it is determined by HPLC that there is an increase in quantity or concentration of a cleavage product of an enzyme relative to a reference quantity or concentration. In some embodiments, it is determined by HPLC that there is a decrease in quantity or concentration of a cleavage product of an enzyme relative to a reference quantity or concentration.

### AAV Vectors for Delivery of Exogenous Nucleic Acids to Target Cells

In some embodiments, nucleic acids encoding enzymes of the methods described herein are incorporated into recombinant AAV (rAAV) vectors and/or virions in order to facilitate their introduction into a cell (e.g., an AAV-permissive cell).

In some embodiments, the AAV-permissive cell is a C2C12 cell, a Huh7 cell, a Hela cell, a HelaS3 cell, a Hepa1-6 cell, a HEK293 cell, a HepG2 cell, or an IMY-N9 cell. For example, in some embodiments, the AAV-permissive cell is a C2C12 cell. In some embodiments, the AAV-permissive cell is a Huh7 cell. In some embodiments, the AAV-permissive cell is a Hela cell. In some embodiments, the AAV-permissive cell is a HelaS3 cell. In some embodiments, the AAV-permissive cell is a Hepa1-6 cell. In some embodiments, the AAV-permissive cell is a HEK293 cell. In some embodiments, the AAV-permissive cell is a HepG2 cell. In some embodiments, the AAV-permissive cell is an IMY-N9 cell. The AAV-permissive cell may be lysed.

rAAV vectors useful in the presently disclosed subject matter are recombinant nucleic acid constructs that include (1) a transgene to be expressed (e.g., a polynucleotide encoding a MTM1 protein) and (2) viral nucleic acids that facilitate integration and expression of the heterologous genes. The viral nucleic acids may include those sequences of AAV that are required in cis for replication and packaging (e.g., functional inverted terminal repeats (ITRs)) of the DNA into a virion. In typical applications, the transgene encodes MTM1, which is useful for correcting a MTM1 mutation in patients suffering from a loss-of-function disease (e.g., a congenital loss-of-function disease), such as XLM™. Such rAAV vectors may also contain marker or reporter genes. Useful rAAV vectors have one or more of the AAV wild type genes deleted in whole or in part but retain functional flanking ITR sequences. The AAV ITRs may be of any serotype (e.g., derived from serotype 2) suitable for a particular application. Methods for using rAAV vectors are described, for example, in Tal et al., J. Biomed. Sci.7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol.76:791-801 (2002) and Bowles et al., J. Virol. 77:423-432 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of MTM1 in muscle cells. rAAV serotype 9 has also been found to be an efficient transducer of muscle cells. Construction and use of AAV vectors and AAV proteins of different serotypes are described, for example, in Chao et al., Mol. Ther.2:619-623 (2000); Davidson et al., Proc. Natl. Acad. Sci. USA 97:3428-3432 (2000); Xiao et al., J. Virol.72:2224-2232 (1998); Halbert et al., J. Virol. 74:1524-1532 (2000); Halbert et al., J. Virol.75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 vector encoding a therapeutic protein pseudotyped with a capsid gene derived from AAV serotype 2. Techniques involving the construction and use of pseudotyped rAAV virions are known in the art and are described, for example, in Duan et al., J. Virol.75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants are described in Wu et al., J. Virol.74:8635-45 (2000). Other rAAV virions that can be used in methods of the presently disclosed subject matter include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

AAV vectors described herein may contain a polynucleotide sequence as well as, e.g., additional sequence elements used for the expression of enzymes and/or the integration of these enzyme sequences into the genome of a mammalian cell. Certain AAV vectors that can be used for the expression of transgenes described herein include regulatory sequences, such as promoter and enhancer regions, which direct gene transcription. Other useful AAV vectors for expression of enzymes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the AAV vector.

In some embodiments of, the promotor is a desmin promoter, a PGK promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3.

In some embodiments, the promoter is a desmin promoter.

The disclosure herein provides a method of producing an AAV encoding an enzyme for treatment of a loss-of-function disease associated with a deleterious mutation in a gene encoding the enzyme in a patient in need thereof, for example by contacting an AAV vector with an AAV-permissive cell expressing a cleavable substrate specific for the enzyme for a time sufficient for the AAV-permissive cell to express the enzyme;

assessing, by way of HPLC, a quantity or concentration of a product resulting from cleavage of the substrate by the enzyme in the cell or surrounding extracellular media;

and releasing the AAV vector for treating the disease if the quantity or concentration of the cleavage product in the cell or surrounding extracellular media is increased relative to a reference quantity or concentration of the cleavage product or decreased relative to a reference quantity or concentration of the cleavage product.

The disclosure herein also provides a method of producing an AAV, for example by contacting an AAV vector with an AAV-permissive cell expressing a cleavable substrate specific for the enzyme for a time sufficient for the AAV-permissive cell to express the enzyme; determining, by way of HPLC, that a quantity or concentration of a product resulting from cleavage of the substrate by the enzyme in the cell or surrounding extracellular media is increased relative to a reference quantity or concentration of the cleavage product or decreased relative to a reference quantity or concentration of the cleavage product; and releasing the AAV vector for treating a loss-of-function disease.

### An Exemplary AAV: Resamirigene Bilparvovec

As described herein, an AAV vector may include a pseudotyped AAV vector including a nucleic acid sequence encoding a MTM1 gene (SEQ ID NO: 3) operably linked to a desmin promotor (SEQ ID NO:1) flanked by AAV2 ITR and packaged within capsid proteins from AAV8 (AAV2/8) as well as the other genetic components listed in Table 2. Said exemplary vector is known by the international proprietary name (INN) of resamirigene bilparvovec.

Resamirigene bilparvovec is a non-replicating recombinant AAV8 vector expressing a non-codon-optimized human MTM1 cDNA under the control of the muscle-specific human desmin promoter. The MTM1 expression cassette was built by cloning a synthetic DNA sequence complementary to the coding portion (nucleotides 43-1864) of the wild-type human MTM1 transcript (NCBI Ref. Seq NM_000252.3) downstream of the 1.05-kb human desmin enhancer/promoter region. The second intron and polyadenylation sequence of the human β-globin gene (HBB) were inserted upstream and downstream respectively of the MTM1 synthetic cDNA to mediate RNA processing. The expression cassette was flanked by AAV serotype-2 (AAV2) inverted terminal repeats (ITRs). The vector was produced in an AAV8 capsid by two-plasmid transfection in HEK293 cells in suspension culture in bioreactors a full GMP process.

The components of resamirigene bilparvovec are shown in Table 2, below:

TABLE 2
Resamirigene Bliparvovec Nucleic Acid Sequence (SEQ ID NO: 2)

| Range | | |
|---|---|---|
| (nucleotides, | | |
| relative to | Length | |
| SEQ ID NO: 2) | (nucleotides) | Genetic Component |
| 3080-3198 | 119 | AAV2 ITR |
| 3199-3256 | 58 | Linker sequence |
| 3257-4316 | 1,060 | Human desmin promoter (SEQ ID NO: 1) |
| 4317-4354 | 38 | Linker sequence |
| 4355-4460 | 106 | Human Beta-globin intron |
| 4373-4848 | 476 | Human Beta-globin intron |
| 4458-4902 | 445 | Human Beta-globin intron |
| 4927-6738 | 1,812 | Human MTM1 coding sequence (SEQ ID |
| | | NO: 3) |
| 6739-6759 | 21 | Linker sequence |
| 6760-7519 | 760 | Human Beta-globin poly-adenylation |
| | | sequence |
| 7520-7551 | 32 | Linker sequence |
| 7552 | 7,696 | AAV2 ITR |

As described herein, resamirigene bilparvovec refers to the AAV vector having the nucleic acid sequence of SEQ ID NO: 2, shown below:

| | |
|---|---|
| TCGCGCGTTT CGGTGATGAC GGTGAAAACC TCTGACACAT GCAGCTCCCG | 50 |
| GACGTCATTG TCGATCCTGC AGGCGTACGG TAAAAAAAGG CATAGCTAAC | 100 |
| AAGGTGTGGA AAAAGAATTA GTGGTTAGAG AGTGAGCTAT TCGTTGAAAC | 150 |
| AATTGCGTTC TTGAAACAAT TCTTGCTGGT AAAATGTCAC ATTTTATGTG | 200 |
| ACTACAGGTG GAGGATTGGC ACATAACCTA ACCAGTGGGG GAAACAATTG | 250 |
| ACCTCTGGAT TTGTCCAAGT GTATAGTAGC ATTTGCCCAA TCGAATGGTC | 300 |
| CTGGTAAGGT GTTAATGTTG ACTAGAACCA AAGGTGGAAG TTGCAGGGAA | 350 |
| ACTGGTTTAG TACAAGGGTG GACACCAGGC AGTCATCCAG AGGCCCATTA | 400 |
| AAGGCCTTGG AATGTTTTTC CGAAGGAGAA TCACTCCCTC TTCTCTCGCT | 450 |
| TAAAGTTTTA GGGGATTCAT GAACAGCTGC TGTGGGATAG TTTCATGTCC | 500 |
| CTAGCAATTG TAAAGCAACT GAGGGTGGCT TAAACCAGTT TTAGCTTTAG | 550 |
| GGTTAGGGTT ACTGGACTAA AATTTGAGAA ATTCATAAAT CTTAAGGAAA | 600 |
| TCCATTGTGA GTTTTCATTA TGAGTGCATC CAATGTATAA TTTCCATGAC | 650 |
| CCTCCCATGC AAGTGAGCAT GTGAATCAGG AAACGTTACA AGAACCCAAC | 700 |
| AAACTCAACC ACTACTAGAC AGGCGATCAC TTCCAGTTAG TATGCAACTT | 750 |
| TCTGTGTAAT TTTAGTTACC ATTAAAATCT GGATGACCTT AGTGTAAGGA | 800 |
| AAAAATACCT TGAATAGTGT TAAAGATGTA CACTTGGTGT CAGGCATTGT | 850 |
| AACATTGATA AATCTGTGTA AGGTGCTTTT TGAAAACTTC AAAGCTGCAT | 900 |
| CAAGTCAAGT ACAAGAAAGG CCATGGCTGC TAAAGCTGTT GAAGATGTGG | 950 |
| GATGGAACTG GGTCACATTG GTGTTAACAG CGTTGTGCAG AGCCGGCAGG | 1000 |
| ATCTTGGTGT GAGCGAACAT TAGTCTATTT AATAAAGCTG TGTGAATGTT | 1050 |
| GTAGAGGTGA GGATGCTCAC TTGAAAACTC ACTGAAGAAC ACTTGGCCCC | 1100 |
| TTGAACTAAA GTGCTTCTAT CAAGTTCAGT GAGAAATTCC GAATTACAAG | 1150 |
| CATAGGTACT AGAAAAGTTT TGAAAAGCAG TATAGAGCAA CATAAGCACA | 1200 |
| TTCATAAAAT TAGTGATGTA GAAAGTGAAA TTTCCACGTA TGGTCACTCC | 1250 |
| CAGAGAAAAA AAATACGTTT ATTTACCTTT TTTAAAAATA GGGGATTTCA | 1300 |
| GGCCGGGTGA GGTGGCTCAC GCCTGTAATC CCAGCACTTT GGGAGGCCCA | 1350 |
| GGTGGGCGGA TCACCTGAGG TCAGGAGTTG GAGGGATGGC AAATCCCATC | 1400 |
| TCTACAAAAT ATACAAAAAA ATAGCTGGGT GTGTTGGCAG GCGCCTGTAA | 1450 |
| TCCCAGCTAC TCGGAAGGCT GAGGCAGGAG AATCCCTGGA ACCAGGGATG | 1500 |
| TGGAGGTTGC AGTGAGCCGA GATTGTGTAA CTGCATTCCA GCCTGGGCAA | 1550 |
| CAAGAGCAAG ACTCCGTATC AGGAAAAAAA AAAGGGGGGG TTGGATTTCG | 1600 |
| CTTGTTGCAT AGGTTGGTCT CAAACTCCTG GCCTCAAGTG ATTCTCCTGC | 1650 |
| CTCTGCCTCC CAAAGTGCTG AGATTACAGG TGTGAGGCAC CATGCCAGGT | 1700 |
| CTCTTACTGT TTGTAATTAA ATACATACAC ATTTTGTGTG TTTGTGTGCA | 1750 |
| CCTTTATAAA GTCAAAGGTG ATAGTAACCC ATTTAAGTTC CTACTCAATT | 1800 |
| TTACTTTCCA GGGATAACTA ACTACTTTTT CTTTTTGAGA TGGAGTCTCG | 1850 |
| CTGTGTAGCC CAGGCTGGAG TGCAGTGGCA CCATCTCGGC TCACTGCAAG | 1900 |
| CTCCTCCTCC CTGGTTCACG CTATTCTCCT GCCTCAGCCT CCCCAACAAC | 1950 |
| TAGGACTACA GGCTCACCTC GCCATACCTG GCTAATTTTT TGTATTTTTA | 2000 |
| GTAGAGACAG GGTTTCACTG TGTTAGCCAG GATGGTCTCG ATCTCCTGAC | 2050 |
| CTTGTGATCC GCCTGCCTCT GCCTCCCAAA GTGCTGGGAT TACAGGCATG | 2100 |
| AGCAACCTCA CCCAGCTGGG ATAACTACTT TTTACAGGTT GATATTCTTT | 2150 |
| TGGACTTTTC CCCTGTGTAA AAATATACTA TATTTGTTAT GTACATATTA | 2200 |
| TGTACATACA GACACAAATT GGACCATTCT CAGTATAATG ATTCTCAGGT | 2250 |
| TTTTTTTTTT TTTTTGAGGT GGGGAACTAG ATAATTATGG ACATCTTTCC | 2300 |
| ATACTAGCAT ATCAATATCT ACCTCATTCT TTTTAATATT TTTGCTAGTA | 2350 |
| TTCCATTGTA TGAATGTCCT ATGATTTACT TAACCTGTCC ATCAATATTT | 2400 |
| GTTTCCAGGT TTTTGCTATT ATAATGCTGC TGCAAAGTAC ATCCTCACAC | 2450 |
| ATCTTTATTT TGTCTATTCA TATTTCTGTA AGATAGGTTA CTAAAGTTGG | 2500 |
| AACTGCCAAA TTAACACTAT CATACTATTT TGTTTTTTAA TTTTAATTTT | 2550 |
| TTAAAAAATG TAAAATGTGC AATTTCAAGA GGAGAAACTT GAACACAAGG | 2600 |
| AGCAAAATCT ATTTTTATAA CATCCTATTA AAAGCTTGCT TTACATAAAG | 2650 |
| ATTTTGAAAG AATAGCATAA ATACAAGATT TCTATTTTAA TTGGATTCTT | 2700 |
| AGGGCTAATA AAATAATCAG CCTTAGCACT TATTTATTTA TTTTTTTTGA | 2750 |
| GAGGGAGTCT CGCTCTGTTG TCCATGCTGG AGTGCAGTGG CGTGATCTCG | 2800 |
| GCTCACTGCA AGCTCCACCT CATGAGTTCA CACCATTCTC CTGCCTCAGT | 2850 |
| CTCCCGAGTA GCTGGGACTC CAGGCGCCCT CTACAAAGCC CGTCTAATTT | 2900 |
| TTTTTGTATT TTTAGTAGAG ACAGGGTTTC ACTGTGTTAG CCAGGATGGT | 2950 |
| CTTGATCTCC TGACCTTGTG ATCTGCCCGC CTCGGCCTCC CAAAGTGCTG | 3000 |
| GGATTATAGG CTTGAGCCAC TGCTCCCGGC CAGCACTTAT TTTTATAATT | 3050 |
| CTTCATGATT ACTGTGTTAC TGTCCCATGG GCCGCCAGGG CCAGCTAGGT | 3100 |
| TGGCCACTCC CTCTCTGCGC GCTCGCTCGC TCACTGAGGC CGGGCGACCA | 3150 |
| AAGGTCGCCC GACGCCCGGG CTTTGCCCGG GCGGCCTCAG TGAGCGAGCG | 3200 |
| AGCGCGCAGA GAGGGAGTGG CCAACTCCAT CACTAGGGGT TCCTCCTAGC | 3250 |
| ACGCGCTACC CCCTGCCCCC CACAGCTCCT CTCCTGTGCC TTGTTTCCCA | 3300 |
| GCCATGCGTT CTCCTCTATA AATACCCGCT CTGGTATTTG GGGTTGGCAG | 3350 |
| CTGTTGCTGC CAGGGAGATG GTTGGGTTGA CATGCGGCTC CTGACAAAAC | 3400 |
| ACAAACCCCT GGTGTGTGTG GGCGTGGGTG GTGTGAGTAG GGGGATGAAT | 3450 |
| CAGGGAGGGG GCGGGGGACC CAGGGGGCAG GAGCCACACA AAGTCTGTGC | 3500 |
| GGGGGTGGGA GCGCACATAG CAATTGGAAA CTGAAAGCTT ATCAGACCCT | 3550 |
| TTCTGGAAAT CAGCCCACTG TTTATAAACT TGAGGCCCCA CCCTCGACAG | 3600 |
| TACCGGGGAG GAAGAGGGCC TGCACTAGTC CAGAGGGAAA CTGAGGCTCA | 3650 |
| GGGCCAGCTC GCCCATAGAC ATACATGGCA GGCAGGCTTT GGCCAGGATC | 3700 |
| CCTCCGCCTG CCAGGCGTCT CCCTGCCCTC CCTTCCTGCC TAGAGACCCC | 3750 |
| CACCCTCAAG CCTGGCTGGT CTTTGCCTGA GACCCAAACC TCTTCGACTT | 3800 |
| CAAGAGAATA TTTAGGAACA AGGTGGTTTA GGGCCTTTCC TGGGAACAGG | 3850 |
| CCTTGACCCT TTAAGAAATG ACCCAAAGTC TCTCCTTGAC CAAAAAGGGG | 3900 |
| ACCCTCAAAC TAAAGGGAAG CCTCTCTTCT GCTGTCTCCC CTGACCCCAC | 3950 |
| TCCCCCCCAC CCCAGGACGA GGAGATAACC AGGGCTGAAA GAGGCCCGCC | 4000 |
| TGGGGGCTGC AGACATGCTT GCTGCCTGCC CTGGCGAAGG ATTGGTAGGC | 4050 |
| TTGCCCGTCA CAGGACCCCC GCTGGCTGAC TCAGGGGCGC AGGCCTCTTG | 4100 |
| CGGGGGAGCT GGCCTCCCCG CCCCCACGGC CACGGGCCGC CCTTTCCTGG | 4150 |
| CAGGACAGCG GGATCTTGCA GCTGTCAGGG GAGGGGAGGC GGGGGCTGAT | 4200 |
| GTCAGGAGGG ATACAAATAG TGCCGACGGC TGGGGGCCCT GTCTCCCCTC | 4250 |
| GCCGCATCCA CTCTCCGGCC GGCCGCCTGC CCGCCGCCTC CTCCGTGCGC | 4300 |
| CCGCCAGCCT CGCCCGGACT CTAGAGGATC CAGATCTAAG CTTCTCTGGT | 4350 |
| CACCGATCCT GAGAACTTCA GGGTGAGTCT ATGGGACCCT TGATGTTTTC | 4400 |
| TTTCCCCTTC TTTTCTATGG TTAAGTTCAT GTCATAGGAA GGGGAGAAGT | 4450 |
| AACAGGGTAC ACATATTGAC CAAATCAGGG TAATTTTGCA TTTGTAATTT | 4500 |
| TAAAAAATGC TTTCTTCTTT TAATATACTT TTTTGTTTAT CTTATTTCTA | 4550 |
| ATACTTTCCC TAATCTCTTT CTTTCAGGGC AATAATGATA CAATGTATCA | 4600 |
| TGCCTCTTTG CACCATTCTA AAGAATAACA GTGATAATTT CTGGGTTAAG | 4650 |
| GCAATAGCAA TATTTCTGCA TATAAATATT TCTGCATATA AATTGTAACT | 4700 |
| GATGTAAGAG GTTTCATATT GCTAATAGCA GCTACAATCC AGCTACCATT | 4750 |
| CTGCTTTTAT TTTATGGTTG GGATAAGGCT GGATTATTCT GAGTCCAAGC | 4800 |
| TAGGCCCTTT TGCTAATCAT GTTCATACCT CTTATCTTCC TCCCACAGCT | 4850 |
| CCTGGGCAAC GTGCTGGTCT GTGTGCTGGC CCATCACTTT GGCAAAGAAT | 4900 |
| TCCGCGGGCG GCCGCAAGTT TCCAGGATGG CTTCTGCATC AACTTCTAAA | 4950 |
| TATAATTCAC ACTCCTTGGA GAATGAGTCT ATTAAGAGGA CGTCTCGAGA | 5000 |
| TGGAGTCAAT CGAGATCTCA CTGAGGCTGT TCCTCGACTT CCAGGAGAAA | 5050 |
| CACTAATCAC TGACAAAGAA GTTATTTACA TATGTCCTTT CAATGGCCCC | 5100 |
| ATTAAGGGAA GAGTTTACAT CACAAATTAT CGTCTTTATT TAAGAAGTTT | 5150 |
| GGAAACGGAT TCTTCTCTAA TACTTGATGT TCCTCTGGGT GTGATCTCGA | 5200 |
| GAATTGAAAA AATGGGAGGC GCGACAAGTA GAGGAGAAAA TTCCTATGGT | 5250 |
| CTAGATATTA CTTGTAAAGA CATGAGAAAC CTGAGGTTCG CTTTGAAACA | 5300 |
| GGAAGGCCAC AGCAGAAGAG ATATGTTTGA GATCCTCACG AGATACGCGT | 5350 |
| TTCCCCTGGC TCACAGTCTG CCATTATTTG CATTTTTAAA TGAAGAAAAG | 5400 |
| TTTAACGTGG ATGGATGGAC AGTTTACAAT CCAGTGGAAG AATACAGGAG | 5450 |
| GCAGGGCTTG CCCAATCACC ATTGGAGAAT AACTTTTATT AATAAGTGCT | 5500 |
| ATGAGCTCTG TGACACTTAC CCTGCTCTTT TGGTGGTTCC GTATCGTGCC | 5550 |
| TCAGATGATG ACCTCCGGAG AGTTGCAACT TTTAGGTCCC GAAATCGAAT | 5600 |
| TCCAGTGCTG TCATGGATTC ATCCAGAAAA TAAGACGGTC ATTGTGCGTT | 5650 |
| GCAGTCAGCC TCTTGTCGGT ATGAGTGGGA AACGAAATAA AGATGATGAG | 5700 |
| AAATATCTCG ATGTTATCAG GGAGACTAAT AAACAAATTT CTAAACTCAC | 5750 |
| CATTTATGAT GCAAGACCCA GCGTAAATGC AGTGGCCAAC AAGGCAACAG | 5800 |
| GAGGAGGATA TGAAAGTGAT GATGCATATC ATAACGCCGA ACTTTTCTTC | 5850 |
| TTAGACATTC ATAATATTCA TGTTATGCGG GAATCTTTAA AAAAAGTGAA | 5900 |
| GGACATTGTT TATCCTAATG TAGAAGAATC TCATTGGTTG TCCAGTTTGG | 5950 |
| AGTCTACTCA TTGGTTAGAA CATATCAAGC TCGTTTTGAC AGGAGCCATT | 6000 |
| CAAGTAGCAG ACAAAGTTTC TTCAGGGAAG AGTTCAGTGC TTGTGCATTG | 6050 |
| CAGTGACGGA TGGGACAGGA CTGCTCAGCT GACATCCTTG GCCATGCTGA | 6100 |
| TGTTGGATAG CTTCTATAGG AGCATTGAAG GGTTCGAAAT ACTGGTACAA | 6150 |
| AAAGAATGGA TAAGTTTTGG ACATAAATTT GCATCTCGAA TAGGTCATGG | 6200 |
| TGATAAAAAC CACACCGATG CTGACCGTTC TCCTATTTTT CTCCAGTTTA | 6250 |
| TTGATTGTGT GTGGCAAATG TCAAAACAGT TCCCTACAGC TTTTGAATTC | 6300 |
| AATGAACAAT TTTTGATTAT AATTTTGGAT CATCTGTATA GTTGCCGATT | 6350 |
| TGGTACTTTC TTATTCAACT GTGAATCTGC TCGAGAAAGA CAGAAGGTTA | 6400 |
| CAGAAAGGAC TGTTTCTTTA TGGTCACTGA TAAACAGTAA TAAAGAAAAA | 6450 |
| TTCAAAAACC CCTTCTATAC TAAAGAAATC AATCGAGTTT TATATCCAGT | 6500 |
| TGCCAGTATG CGTCACTTGG AACTCTGGGT GAATTACTAC ATTAGATGGA | 6550 |
| ACCCCAGGAT CAAGCAACAA CAGCCGAATC CAGTGGAGCA GCGTTACATG | 6600 |
| GAGCTCTTAG CCTTACGCGA CGAATACATA AAGCGGCTTG AGGAACTGCA | 6650 |
| GCTCGCCAAC TCTGCCAAGC TTTCTGATCC CCCAACTTCA CCTTCCAGTC | 6700 |
| CTTCGCAAAT GATGCCCCAT GTGCAAACTC ACTTCTGACC GGTCCGAGGG | 6750 |
| CCCAGATCTA ATTCACCCCA CCAGTGCAGG CTGCCTATCA GAAAGTGGTG | 6800 |
| GCTGGTGTGG CTAATGCCCT GGCCCACAAG TATCACTAAG CTCGCTTTCT | 6850 |
| TGCTGTCCAA TTTCTATTAA AGGTTCCTTT GTTCCCTAAG TCCAACTACT | 6900 |
| AAACTGGGGG ATATTATGAA GGGCCTTGAG CATCTGGATT CTGCCTAATA | 6950 |
| AAAAACATTT ATTTTCATTG CAATGATGTA TTTAAATTAT TTCTGAATAT | 7000 |
| TTTACTAAAA AGGGAATGTG GGAGGTCAGT GCATTTAAAA CATAAAGAAA | 7050 |
| TGAAGAGCTA GTTCAAACCT TGGGAAAATA CACTATATCT TAAACTCCAT | 7100 |
| GAAAGAAGGT GAGGCTGCAA ACAGCTAATG CACATTGGCA ACAGCCCCTG | 7150 |
| ATGCCTATGC CTTATTCATC CCTCAGAAAA GGATTCAAGT AGAGGCTTGA | 7200 |
| TTTGGAGGTT AAAGTTTTGC TATGCTGTAT TTTACATTAC TTATTGTTTT | 7250 |
| AGCTGTCCTC ATGAATGTCT TTTCACTACC CATTTGCTTA TCCTGCATCT | 7300 |
| CTCAGCCTTG ACTCCACTCA GTTCTCTTGC TTAGAGATAC CACCTTTCCC | 7350 |
| CTGAAGTGTT CCTTCCATGT TTTACGGCGA GATGGTTTCT CCTCGCCTGG | 7400 |
| CCACTCAGCC TTAGTTGTCT CTGTTGTCTT ATAGAGGTCT ACTTGAAGAA | 7450 |
| GGAAAAACAG GGGGCATGGT TTGACTGTCC TGTGAGCCCT TCTTCCCTGC | 7500 |
| CTCCCCCACT CACAGTGACC GGCCGCTCTA GGAGGAACCC CTAGTGATGG | 7550 |
| AGTTGGCCAC TCCCTCTCTG CGCGCTCGCT CGCTCACTGA GGCCGGGCGA | 7600 |
| CCAAAGGTCG CCCGACGCCC GGGCTTTGCC CGGGCGGCCT CAGTGAGCGA | 7650 |
| GCGAGCGCGC AGAGAGGGAG TGGCCAACCT AGAGGCCGCC AGGGCCATAT | 7700 |
| TTCTCAATTT TTAAATTTTT CAAAAAAATT AATCCTTAAT GTGCATATTT | 7750 |
| TTGAATTGTT AATATAACTT TTTGAGGTGA TGTCTTCATG TGTTTCAACT | 7800 |
| ACTTAAAAAC TTTTAAACAG TATATAATAA AAAATCTTCC AGGCCACTCA | 7850 |
| CACCTGTAAT CCCAGCACTT TGGGAGGCTG AGGTGGGCAG ATCACCTGAG | 7900 |
| GGCAGGAGTT CGAGACCAGC CTGGCCAATA TATATATATT CATATATTCA | 7950 |
| TATATATATA TATATTCATA TATTCATATA TATATATTCA TATATTCATA | 8000 |
| TATATATATA TATATATATA TAGCAAAACC TCATCTCTAA TAAAATACAA | 8050 |
| AAATTAGCTG AGCGTGGTGA TGGATGCCTG TAGTCCCAGC TACTCGGGAG | 8100 |
| GCTGAGGCAG GAGAATCTCT TGAACCTGGG AGGTGGAGGT TGCAGTGAGC | 8150 |
| TGAGATGGTG CCACTGCCCT CCAGCCTGAG TGACAGAGCG AGACTCGGTC | 8200 |
| TCCAAAAAAA AACAACAAAA AAATCTTCCA TCCTTGTCTC CCATCCACCC | 8250 |
| CTTCCCCCCA GCATGTACTT GCAGACTTTA TGCATATACA GTGAGTACTG | 8300 |
| TATATACACA AATAATAAAA AAATCATATA TATAATATAT GTAATTCCCC | 8350 |
| TTTACATGAA AGGTAGCACA CTGGTCTGTA CAGTCTGTCT GCACTGTGCT | 8400 |
| ATTTCACTTT ATATTTTTAT AGTTTGACAG AGTTCTAACA TTTCTTTTTT | 8450 |
| TTTTTTTTTA ACAGAGTCTT GTTCCTGATT GTTAAATTTT AAAGCATCCT | 8500 |
| AAAGTTTGGT TTCACACTTG AATGAATACC ATGTAAGGAT TCACTTACAT | 8550 |
| AGATGTGGTT GCCTGAATCT TAAGAATAAA ATAACATTGT TTGTATTTAT | 8600 |
| TTAAATTAGT GTTCCTTTTA TGGTTTGCCT GAAAGCACAA CAAAATCCTC | 8650 |
| ACCAAGATAT TACAATTATG ACTCCCATAC AGGTAAACTG TTTAGAGATT | 8700 |
| GGCAAGCACC TTTTAATGAA AGGAGTCAGC CAGCTTAGTG TGCAGTATTT | 8750 |
| ATTTCTGCCG GAAGAGGGAG CTTCAGGGAC AGACTTTGGT TTAGTCATGA | 8800 |
| AGCCTCCAGC ACTCCCAAGC GGTTGTGGTT GACCAAGCAA TTTATGCTTT | 8850 |
| TACCTTTCTA CTTCCAGAGG CTTGTTTACT TATCAGTAAG CATTAATTTA | 8900 |
| GTGTCCCCTC AGATGCCTTT TACTTTCTTC TTTTCTGCCT AGAATAAGCT | 8950 |
| GCTCTTCCAA TTTTGCAGCT ACATGTTTCC ACCCCAGTTG GAATTTCTCC | 9000 |
| ATAACATCCA TTGTAGCTAT CCTTCAATCT ACAGCCTCTA TTTCCTGTTA | 9050 |
| TAGCTGGTCA GGTCTAATCC CTCAAAATAC TCTGTCCCCT GCTTCCCTTA | 9100 |
| TCTGCTGGCC ACCTTTTTCC CCCACATACA CACTGCCATG TCCCACCCTT | 9150 |
| CACTCAAGTT GTTCCCTGCC ACCTCAACAA ATTTAAGTCC ATAAAATAGA | 9200 |
| GTAAGTGTTC CTGACTGTTA AATTTTAAAG CATCCCAAAG TCTGATTTCA | 9250 |
| CACTCGAATG AATACTATGT ACGGATTCAT TTACATAGAT GCGGTTGCAT | 9300 |
| GAGTCTTAAC AAAAAAATAA CATTATTTGT ATTTATTCAA AGTACTGTCA | 9350 |
| AGATATAATG TCAAGACCTA ATTCAAAGGT TCCACAAAGC CTTCCTTGAC | 9400 |
| TGCCCCCAAC GAAGATTATC CATTTTCCCT GAAATCCCAT TGACTTTTCT | 9450 |
| ATTTTGTAAG GAGGCTCGTG AGACTCTGTC TAAAAACAAA ACAAAACAAA | 9500 |
| AAGAAACAAT CAAACGGCTT GCTTCTGTTC TTTGATCTGC TAGTAAGCAA | 9550 |
| AAATTACACA TGGTGACAGG AGCTATGTGA GGCTGTCAGG TTGAATGGGA | 9600 |
| GGAGTTTGGG ATCCTGCTTG TGGATGGTTG GAAGAGGCTT TCGGGAAAGA | 9650 |
| CAGTATTTAT GTGAGACCTG GAAGATGGGC CTTAGCTTTG CAGAAGGTGG | 9700 |
| AGAGGCAGGA AATAGCACGG GGGCCCTGGG GCTGGAAGAC TTGGGCATAT | 9750 |
| TTGAGGAACA GAAAGGAGAC CAGCATAACT GAGGTGGGAA AAGCATGTGA | 9800 |
| AGAGATGGGG CTGGAGGAGG CCGGGAGTGG TGGCTCACGC CTGTAATCCC | 9850 |
| AGCACTTTGG GAGGCCAAGG CAGGCGGATC ATGAGCTCAG GAGATTGAGA | 9900 |
| CCATCCTGGC TAACACGGTG AAACCCCCTC TCTACTAAAA ATACAAAAAA | 9950 |
| AAAAAAAAAA AAAATTAGCT GGGCGTGGTG GCAGGAGCCT GTAGTCCCAG | 10000 |
| CTACCTGGGA GGCTGAGGCA GGAGAATGGC GTGAACCTGG AAGGCTGAGC | 10050 |
| TTGCAGTGAG CCGAGATTGC ACCACTGCAC TCCAGCCTGG GAGACAGAGA | 10100 |
| GAGACTCCCT CTCAAAAAAA CAAACAAACG AAACAAAACA AAACAAAAAT | 10150 |
| TAGCCAGGCG TGGTGGTATG CACCTGTAAT CCCAGCTACT CGGGAGGTTG | 10200 |
| AGGCAGGAGA AACGCTTGAA CTCAGGAGGC GGAGGTTGCA GTGAGCCGAG | 10250 |
| ACTGCGCCAC TGCACTCCAG CCTGGGTGAC AGAGGGAGAC TCCATCTCAA | 10300 |
| AAAAAAAAAT TTTTTTTTTT TTACAAACGG TGTCTCCCTC TGTCGCCCAG | 10350 |
| GCTGGAGTGC AGTGGTGTGA TCACAGCTCA CTCCAGCCTC AACCTCCCCA | 10400 |
| GCTGAAGCCA TCCTCTTGCC TCAGCCTCCT AAGTAGCTGG GACTACAGGC | 10450 |
| GCGCACCTCC AGGCTTGGCT CTTATTCTTT TTATTGTTTT TGAAACTATA | 10500 |
| GAACCTATTT TTAAAAAATG TTTTGGTTGT TTTTATTGCT GCTTTTCCTT | 10550 |
| TTGGGGTTAG AACACAAGTT TTGATGGGAA ACAGGTTAGA ACACATTCAT | 10600 |
| CTCTTCCCAT AGCGATGGTC ATAGAAAAAC GGGGCATATT TATAAACTCT | 10650 |
| CAGTTGATCT TAAAATGTGC AAAAGCTGCC GAACTCCTGG GAGTGAGCTC | 10700 |
| GAGCCCTGCA GGATCATTGT CACATGTGAG CAAAAGGCCA GCAAAAGGCC | 10750 |
| AGGAACCGTA AAAAGGCCGC GTTGCTGGCG TTTTTCCATA GGCTCCGCCC | 10800 |
| CCCTGACGAG CATCACAAAA ATCGACGCTC AAGTCAGAGG TGGCGAAACC | 10850 |
| CGACAGGACT ATAAAGATAC CAGGCGTTTC CCCCTGGAAG CTCCCTCGTG | 10900 |
| CGCTCTCCTG TTCCGACCCT GCCGCTTACC GGATACCTGT CCGCCTTTCT | 10950 |
| CCCTTCGGGA AGCGTGGCGC TTTCTCATAG CTCACGCTGT AGGTATCTCA | 11000 |
| GTTCGGTGTA GGTCGTTCGC TCCAAGCTGG GCTGTGTGCA CGAACCCCCC | 11050 |
| GTTCAGCCCG ACCGCTGCGC CTTATCCGGT AACTATCGTC TTGAGTCCAA | 11100 |
| CCCGGTAAGA CACGACTTAT CGCCACTGGC AGCAGCCACT GGTAACAGGA | 11150 |
| TTAGCAGAGC GAGGTATGTA GGCGGTGCTA CAGAGTTCTT GAAGTGGTGG | 11200 |
| CCTAACTACG GCTACACTAG AAGAACAGTA TTTGGTATCT GCGCTCTGCT | 11250 |
| GAAGCCAGTT ACCTTCGGAA AAAGAGTTGG TAGCTCTTGA TCCGGCAAAC | 11300 |
| AAACCACCGC TGGTAGCGGT GGTTTTTTTG TTTGCAAGCA GCAGATTACG | 11350 |
| CGCAGAAAAA AAGGATCTCA AGAAGATCCT TTGATCTTTT CTACGGGGTC | 11400 |
| TGACGCTCAG TGGAACGAAA ACTCACGTTA AGGGATTTTG GTCATGAGAT | 11450 |
| TATCAAAAAG GATCTTCACC TAGATCCTTT TAAATTAAAA ATGAAGTTTT | 11500 |
| AAATCAAGCC CAATCTGAAT AATGTTACAA CCAATTAACC AATTCTGATT | 11550 |
| AGAAAAACTC ATCGAGCATC AAATGAAACT GCAATTTATT CATATCAGGA | 11600 |
| TTATCAATAC CATATTTTTG AAAAAGCCGT TTCTGTAATG AAGGAGAAAA | 11650 |
| CTCACCGAGG CAGTTCCATA GGATGGCAAG ATCCTGGTAT CGGTCTGCGA | 11700 |
| TTCCGACTCG TCCAACATCA ATACAACCTA TTAATTTCCC CTCGTCAAAA | 11750 |
| ATAAGGTTAT CAAGTGAGAA ATCACCATGA GTGACGACTG AATCCGGTGA | 11800 |
| GAATGGCAAA AGTTTATGCA TTTCTTTCCA GACTTGTTCA ACAGGCCAGC | 11850 |
| CATTACGCTC GTCATCAAAA TCACTCGCAT CAACCAAACC GTTATTCATT | 11900 |
| CGTGATTGCG CCTGAGCGAG ACGAAATACG CGATCGCTGT TAAAAGGACA | 11950 |
| ATTACAAACA GGAATCGAAT GCAACCGGCG CAGGAACACT GCCAGCGCAT | 12000 |
| CAACAATATT TTCACCTGAA TCAGGATATT CTTCTAATAC CTGGAATGCT | 12050 |
| GTTTTTCCGG GGATCGCAGT GGTGAGTAAC CATGCATCAT CAGGAGTACG | 12100 |
| GATAAAATGC TTGATGGTCG GAAGAGGCAT AAATTCCGTC AGCCAGTTTA | 12150 |
| GTCTGACCAT CTCATCTGTA ACATCATTGG CAACGCTACC TTTGCCATGT | 12200 |
| TTCAGAAACA ACTCTGGCGC ATCGGGCTTC CCATACAAGC GATAGATTGT | 12250 |
| CGCACCTGAT TGCCCGACAT TATCGCGAGC CCATTTATAC CCATATAAAT | 12300 |
| CAGCATCCAT GTTGGAATTT AATCGCGGCC TCGACGTTTC CCGTTGAATA | 12350 |
| TGGCTCATAA CACCCCTTGT ATTACTGTTT ATGTAAGCAG ACAGTTTTAT | 12400 |
| TGTTCATGAT GATATATTTT TATCTTGTGC AATGTAACAT CAGAGATTTT | 12450 |
| GAGACACGGG CCAGAGCTGC A | 12500 |

### Methods for the Delivery of Exogenous Nucleic Acids to Target Cells

### Transfection Techniques

Techniques that can be used to introduce an AAV vector, such as an AAV vector encoding an enzyme described herein, into a target cell are known in the art. For example, electroporation can be used to permeabilize mammalian cells (e.g., human target cells) by the application of an electrostatic potential to the cell of interest.

Mammalian cells, such as human cells, subjected to an external electric field in this manner are subsequently predisposed to the uptake of exogenous nucleic acids (e.g., nucleic acids capable of expression in e.g., neurons, glial cells, or non-neural cells, such as colon and kidney cells). Electroporation of mammalian cells is described in detail, e.g., in Chu et al., Nucleic Acids Research 15:1311 (1987), the disclosure of which is incorporated herein by reference. A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., J. Vis. Exp.81: e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Top. Curr. Chem.228:227 (2003), the disclosure of which is incorporated herein by reference) polyethylenimine, and DEAE-dextran, the use of which as a transfection agent is described in detail, for example, in Gulick et al., Curr Protoc Mol Biol.40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:251870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Ther.9:102 (2002), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods Cell Biol.82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA: DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nat. Rev. Genet. 11:636 (2010); and in Joung et al., Nat. Rev. Mol. Cell Biol. 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Pharmaceutical Compositions and Routes of Administration

The AAV vectors described herein may encode an enzyme, such as an AAV encoding MTM1 which may be incorporated into a vehicle for administration into a patient, such as a human patient suffering from a congenital loss-of-function disease (for example, XLM™). Pharmaceutical compositions containing viral vectors, that contain the transcription regulatory elements (e.g., a desmin promoter) described herein operably linked to a therapeutic enzyme can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

AAV vectors described herein, containing the transcription regulatory element operably linked to a therapeutic enzyme may be administered to a patient (e.g., a human patient) by a variety of routes of administration. The route of administration may vary, for example, with the onset and severity of disease, and may include, e.g., intradermal, transdermal, parenteral, intravenous, intramuscular, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Mixtures of the AAV vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

In some embodiments, upon administering the AAV vector to the patient, the patient displays a change from baseline in quantitative analysis of expression of the enzyme encoded by the AAV (e.g., in a muscle biopsy). In some embodiments, the patient displays the change from baseline in quantitative analysis of the enzyme expression in a muscle biopsy by about 24 weeks after administration of the AAV vector to the patient.

### Kits

The compositions described herein can be provided in a kit for determining the efficacy of an AAV vector in accordance with the methods described herein. In some embodiments, the kit may include one or more AAV vectors encoding an enzyme as described herein. The kit can include a package insert that instructs a user of the kit, such as a scientist of skill in the art, to perform any one of the methods described herein.

### EXAMPLES

The following examples are put forth so as to provide those of ordinary skill in the art with a description of how the compositions and methods described herein may be used and evaluated and are intended to be purely exemplary of the presently disclosed subject matter and are not intended to limit the scope of what the inventors regard as their presently disclosed subject matter.

### Example 1. Viral Vector Encoding an Enzyme Activity Assay

The objective of this study was to examine the efficacy of an adeno-associated virus (AAV) vector encoding an enzyme (e.g., a phosphatase), as determined by the amount or concentration of substrate cleaved by the enzyme (e.g., phosphatase activity) encoded by said AAV relative to a reference quantity or concentration of the cleavage product using a novel potency assay protocol.

### Materials and Methods

### Infection of AAV to Host Cells and Preparation of Cell Lysates

On Day 0, seed host cells (e.g., C2C12 cells; ATCC® CRL1772) to collagen coated 24 well clear plate (50,000 cells/0.5 mL/well) in growth medium (e.g., DMEM, 20% FBS, Penicillin-Streptomycin). Incubate at 37° C., 5% CO₂. On Day 1, first discard medium and add 0.5 mL of differentiation medium (e.g., DMEM, 2% FBS, Penicillin-Streptomycin) to each well. Second, add from 5×10⁹ vg/well to 5×10¹¹ vg/well of an AAV vector encoding an enzyme (e.g., resamirigene bilparvovec). Third, incubate at 37° C., 5% CO₂ for 3 days. On Day 4, first discard medium and wash with PBS. Following, add 500 μL of Lysis Buffer* to each well and pipetting. Lysis buffer may include 50 mM Tris-HCl (pH 8.0), 100 mM NaCl, 10% Glycerol, 5 mM EDTA, 0.5% Triton X-100, 1 mM Dithiothreitol, and 1× Halt™ Protease Inhibitor Cocktail (Thermo Fisher #78437). Afterwards, transfer cell lysates to a 1.5 mL tube. Following, centrifuge at 15,000 rpm, 4° C., for 10 min, and transfer supernatant to new 1.5 mL tube (e.g., dispense to two or three aliquots) and store at −80° C.

### Enzyme Cleavage Assay

First, thaw tubes and measure protein concentrations of the samples by Pierce BCA Protein Assay kit (Thermo Fisher #23227). This assay includes the steps of adding 10 μL of standards and samples to a 96 well plate. Next, add 10 μL of lysis buffer to standard wells. In some embodiments, different formulations of the lysis buffer affect the results. Followingly, add 200 μL of BCA working solution (A: B=1:50) to each well. Shake for 30 seconds, incubate at 37° C. for 30 min, shake again for 30 seconds, and measure the optical density at 562 nm.

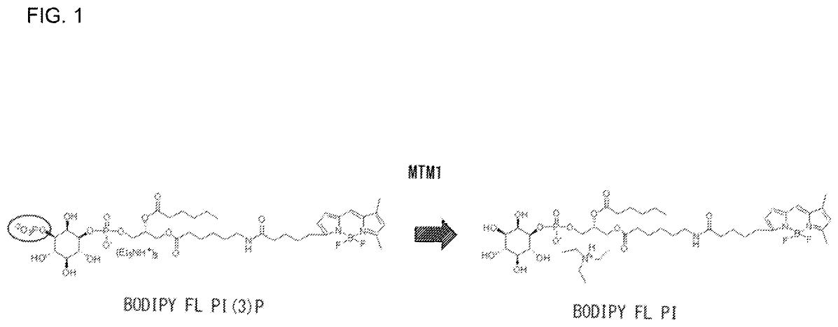

Add substrates and lysates to 1.5 mL tubes. In particular, this includes 50 mM ammonium acetate (pH 6.0), 100 ng BODIPY FL phosphatidylinositol 3-phosphate (hereinafter “BODIPY FL PI (3) P”; FIG. 1) (Echelon Bioscience #C-03F6), 10 μL cell lysate (300 ng, cell lysates are diluted with Lysis Buffer) for a total reaction volume of 100 μL.

Following, incubate at 30° C. for 60 minutes. Then add 2 μL of 10% SDS (for a final concentration of 0.2%) and incubate at 75° C. for 10 minutes. The purpose of this step is to inactivate the AAV. During incubation, prepare standards in 1.5 mL tubes. This would include 0.3 ng to 100 ng of BODIPY FL PI (3) P, a water-soluble analog of PI (3) P labeled with the green fluorophore, Bodipy®-FL at the sn-1 position, and Bodipy®-FL (Echelon Bioscience #C-00F6) in 100 μL of 50 mM ammonium acetate (pH 6.0), 0.1× lysis buffer, and 0.2% SDS.

### Extraction of Cleavage Product

First, add 100 μL chloroform/methanol (1:1) to samples and standards. Afterwards, vortex and centrifuge at 15,000 rpm at 25° C. for 5 min. Followingly, 120 μL of fluorescent lipids (upper phase) are collected and transfer to a 96 deep well plate (BM EQUIPMENT #BM6019). These collections are dried by gas (e.g., N2 gas) blowing. The samples can be stored at −20° C.

### High Performance Liquid Chromatography

### A. Sample Preparation

Before analysis, the dried samples were reconstituted in 50 μL of the starting LC eluent Mobile phase A/B (25:75 v/v), followed by mixing and 10 min centrifugation (3,000 rpm) at 4° C. The supernatants were transferred into an autosampler vials.

### B. Equipment and Conditions

Equipment and conditions included: HPLC of Acquity UPLC H-Class Bio (WATERS), Bio-inert, a Acquity UPLC FLR (Fluorescence) (WATERS) Detector, the λex/Nem was 358/505 (nm) with a gain of 1, a column consisting of L-column2 HPLC Column, 2.0×150 mm, 3 μm (CERI #731021), Mobile phase A consisted of 5 mM Ammonium formate/Acetonitrile=70/30, while Mobile phase B consisted of 2-Propanol/Acetonitrile=9/1, an injection volume of 1 μL, and a run time of 10 minutes.

### C. Liquid Chromatography Conditions:

In particular, the liquid chromatography binary gradient conditions, as described in Table 3, were as follow:

TABLE 3
Liquid Chromatography Binary Gradient Conditions

| Time (min) | Flow (μL/min) | % A | % B |
|---|---|---|---|
| 0 | 0.2 | 25 | 75 |
| 4 | 0.2 | 25 | 75 |
| 6 | 0.2 | 95 | 5 |
| 7.5 | 0.4 | 95 | 5 |
| 7.6 | 0.4 | 25 | 75 |
| 10 | 0.4 | 25 | 75 |
| 10.1 | 0.2 | 25 | 75 |

### Results

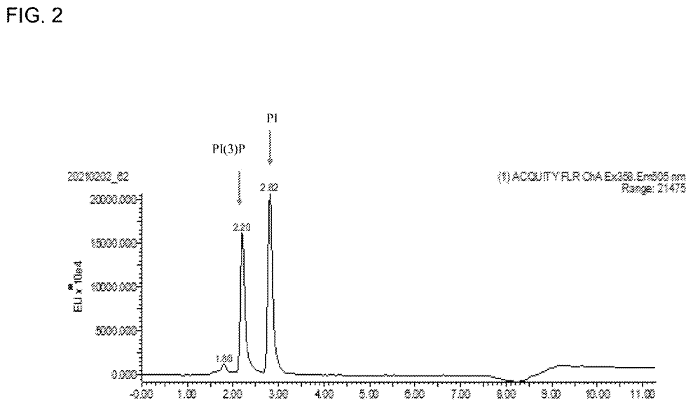

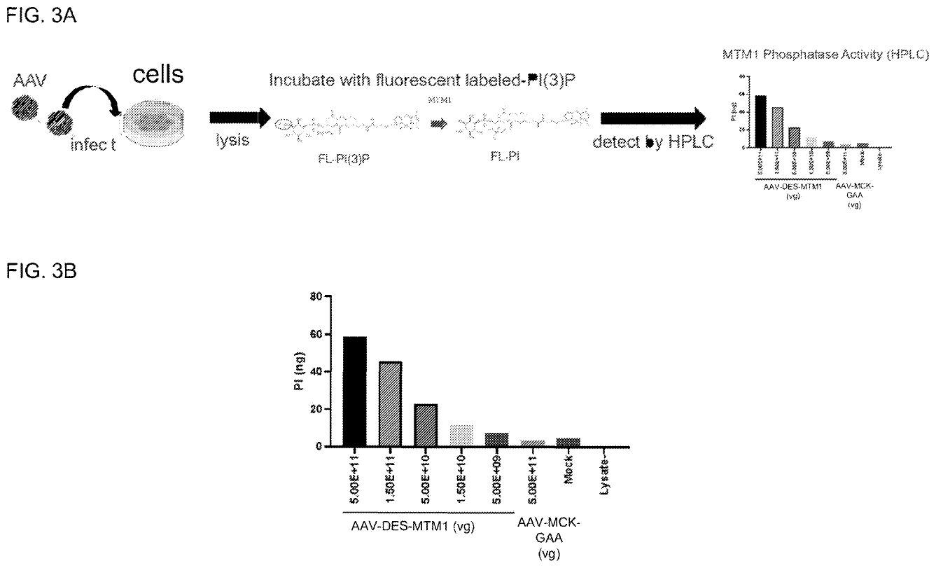

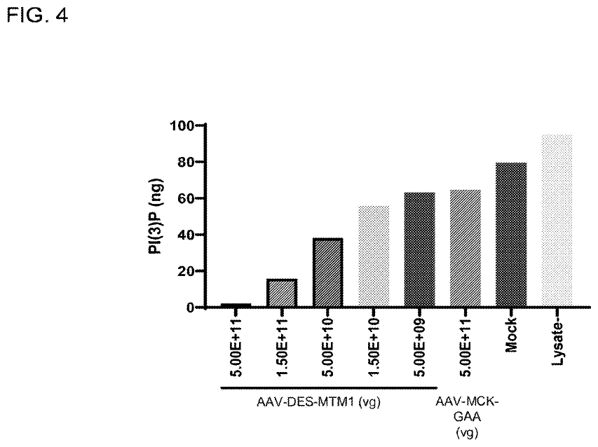

In result, the chromatogram evidenced extraction of both PI (3) P and PI on the chloroform/methanol column (FIG. 2). The quantity (ng) of extracted fluorescently labelled (FL) phosphatidylinositol (PI) increased as a function of increasing the titer of an AAV encoding MTM1 (AAV-DES-MTM1) to C2C12-transfected cells. In contrast, when an AAV encoding acid alpha-glucosidase (GAA: AAV-MCK-GAA) was transfected to C2C12 cells, the quantity (ng) of extracted phosphatidylinositol 3-phosphate (PI (3) P) was significantly reduced (FIG. 3). In line with these results, the quantity (ng) of extracted PI (3) P decreased as a function of increasing the titer of the AAV encoding MTM1 (AAV-DES-MTM1) to C2C12-transfected cells (FIG. 4).

Together, these results illustrate the development of a novel AAV potency assay that provides high-throughput, quantitative, and high detection sensitivity measurements of the potency of AAVs encoding an enzyme (e.g., MTM1).

### Example 2. AAV Encoding Myotubularin 1 Potency Assay

This example describes a method of determining the efficacy of an AAV vector encoding MTM1 for treatment of a loss-of-function disease (e.g., X-linked myotubular myopathy (XLM™) associated with a deleterious mutation in a gene encoding the enzyme in a patient.

According to the Materials and Methods described in Example 1, an AAV vector encoding an enzyme is contacted with an AAV-permissive cell (e.g., a C2C12 cell) expressing a cleavable substrate specific for the enzyme for a time sufficient for the AAV-permissive cell to express the enzyme. An exemplary AAV vector for transfection is resamirigene bilparvovec, which is a non-replicating recombinant AAV8 vector expressing a non-codon-optimized human MTM1 cDNA under the control of the muscle-specific human desmin promoter. The MTM1 expression cassette was built by cloning a synthetic DNA sequence complementary to the coding portion (nucleotides 43-1864) of the wild-type human MTM1 transcript (NCBI Ref. Seq NM_000252.3) downstream of the 1.05 kb human desmin enhancer/promoter region. The second intron and polyadenylation sequence of the human β-globin gene (HBB) were inserted upstream and downstream respectively of the MTM1 synthetic cDNA to mediate RNA processing. The expression cassette was flanked by AAV2 inverted terminal repeats (ITRs). The vector was produced in an AAV8 capsid by two-plasmid transfection in HEK293 cells in suspension culture in bioreactors a full GMP process.

Afterwards, by way of HPLC, a quantity or concentration of a product resulting from cleavage of the substrate by the MTM1 enzyme in surrounding extracellular media can be assessed.

According to the Materials and Methods described in Example 1, a finding that the quantity or concentration of FL-PI, the cleavage product of MTM1, in the cell or surrounding extracellular media is increased relative to a reference quantity or concentration of the FL-PI, identifies the resamirigene bilparvovec as being efficacious for treating the loss-of-function disease (e.g., XLM™).

### Example 3. A Method of Treating a Loss-of-Function Disease

This example describes a method of treating a loss-of-function disease associated with a deleterious mutation in a gene encoding an enzyme in a patient in need thereof, including determining the efficacy of an AAV vector encoding the putative enzyme and administering a therapeutically effective amount of the AAV vector the patient.

According to the Materials and Methods described in Example 1, an AAV vector encoding an enzyme is contacted with an AAV-permissive cell (e.g., a C2C12 cell) expressing a cleavable substrate specific for the enzyme for a time sufficient for the AAV-permissive cell to express the enzyme. An exemplary AAV vector for transfection is resamirigene bilparvovec, which is a non-replicating recombinant AAV8 vector expressing a non-codon-optimized human MTM1 cDNA under the control of the muscle-specific human desmin promoter. The MTM1 expression cassette was built by cloning a synthetic DNA sequence complementary to the coding portion (nucleotides 43-1864) of the wild-type human MTM1 transcript (NCBI Ref. Seq NM_000252.3) downstream of the 1.05 kb human desmin enhancer/promoter region. The second intron and polyadenylation sequence of the human β-globin gene (HBB) were inserted upstream and downstream respectively of the MTM1 synthetic cDNA to mediate RNA processing. The expression cassette was flanked by AAV2 inverted terminal repeats (ITRs). The vector was produced in an AAV8 capsid by two-plasmid transfection in HEK293 cells in suspension culture in bioreactors a full GMP process.

Afterwards, by way of HPLC, a quantity or concentration of a product resulting from cleavage of the substrate by the enzyme in surrounding extracellular media can be assessed. A finding that the quantity or concentration of FL-PI, the cleavage product of MTM1, in the cell or surrounding extracellular media is increased relative to a reference quantity or concentration of the FL-PI, identifies the AAV vector as being efficacious for treating the loss-of-function disease.

After determining that the quantity or concentration of the cleavage product in the cell or surrounding extracellular media is increased relative to a reference quantity or concentration of the of the cleavage product the patient is administered a therapeutically effective amount of the AAV vector (e.g., in an amount of from about 3×10¹³ vg/kg to about 2.3×10¹⁴ vg/kg, optionally wherein the AAV vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg).

### OTHER EMBODIMENTS

All publications, patents, and patent applications mentioned in this specification are incorporated herein by reference to the same extent as if each independent publication or patent application was specifically and individually indicated to be incorporated by reference.

While the presently disclosed subject matter has been described in connection with specific embodiments thereof, it will be understood that it is capable of further modifications and this application is intended to cover any variations, uses, or adaptations of the presently disclosed subject matter following, in general, the principles of the presently disclosed subject matter and including such departures from the presently disclosed subject matter that come within known or customary practice within the art to which the presently disclosed subject matter pertains and may be applied to the essential features hereinbefore set forth, and follows in the scope of the claims.

Other embodiments are within the claims.

1. A method of determining the efficacy of an adeno-associated virus (AAV) vector encoding an enzyme for treatment of a loss-of-function disease associated with a deleterious mutation in a gene encoding the enzyme in a patient, the method comprising:
a) contacting the AAV vector with an AAV-permissive cell for a time sufficient for the AAV-permissive cell to express the enzyme;
b) lysing the cell and exposing the expressed enzyme, in a reaction medium, to a cleavable substrate specific for the enzyme; and
c) assessing, by way of chromatography, a quantity or concentration of the substrate or of a product resulting from cleavage of the substrate by the enzyme in the reaction medium,
wherein a finding that (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate identifies the AAV vector as being efficacious for treating the disease.

2. A method of producing an AAV vector encoding an enzyme for treatment of a loss-of-function disease associated with a deleterious mutation in a gene encoding the enzyme in a patient in need thereof, the method comprising:
a) contacting the AAV vector with an AAV-permissive cell for a time sufficient for the AAV-permissive cell to express the enzyme;
b) lysing the cell and exposing the expressed enzyme, in a reaction medium, to a cleavable substrate specific for the enzyme;
c) assessing, by way of chromatography, a quantity or concentration of the substrate or of a product resulting from cleavage of the substrate by the enzyme in the reaction medium; and
d) releasing the AAV vector for treating the disease if (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate.

3. (canceled)

4. A method of treating a loss-of-function disease associated with a deleterious mutation in a gene encoding an enzyme in a patient in need thereof, the method comprising:
a) contacting the AAV vector with an AAV-permissive cell for a time sufficient for the AAV-permissive cell to express the enzyme;
b) lysing the cell and exposing the expressed enzyme, in a reaction medium, to a cleavable substrate specific for the enzyme;
c) determining, by way of chromatography, that (i) the quantity or concentration of the cleavage product in the reaction medium is increased relative to a reference quantity or concentration of the cleavage product or (ii) the quantity or concentration of the substrate in the reaction medium is decreased relative to a reference quantity or concentration of the substrate; and
d) administering a therapeutically effective amount of the AAV vector to the patient.

5. (canceled)

6. The method of claim 1, wherein the expressed enzyme is not purified from cell lysate resulting from (b) prior to exposing the enzyme to the cleavable substrate.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The method of claim 1, wherein the enzyme is Myotubularin 1 (MTM1).

31. (canceled)

32. (canceled)

33. (canceled)

34. (canceled)

35. The method of claim 1, wherein the AAV vector further comprises a muscle specific promoter, optionally wherein the promoter is positioned 5′ to a polynucleotide encoding the enzyme.

36. The method of claim 35, wherein the muscle specific promotor is a desmin promoter, a phosphoglycerate kinase promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The method of claim 1, wherein the AAV vector has a nucleic acid sequence that is at least 85% identical to the nucleic acid sequence of SEQ ID NO: 2.

49. (canceled)

50. (canceled)

51. (canceled)

52. (canceled)

53. (canceled)

54. (canceled)

55. (canceled)

56. The method of claim 4, wherein the AAV vector is administered to the patient in an amount of from about 3×10¹³ vg/kg to about 2.3×10¹⁴ vg/kg, optionally wherein the AAV vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg.

57. (canceled)

58. (canceled)

59. (canceled)

60. (canceled)

61. (canceled)

62. (canceled)

63. (canceled)

64. (canceled)

65. (canceled)

66. The method of claim 5665, wherein the congenital loss-of-function disease is X-linked myotubular myopathy (XLM™).

67. A kit comprising an AAV vector encoding an enzyme and a package insert, wherein the package insert instructs a user of the kit to determine the efficacy of the AAV vector in accordance with the method of claim 66.


SEQUENCE LISTING (4 sequences)

SEQ ID NO: 1 - DNA, 1060 bp, synthetic construct
  source 1..1060: mol_type = other DNA
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 2 - DNA, 12471 bp, synthetic construct
  source 1..12471: mol_type = other DNA
tcgcgcgtttcggtgatgacggtgaaaacctctgacacatgcagctcccggacgtcattgtcgatcctgcaggcgtacggtaaaaaaaggcatagctaacaaggtgtggaaaaagaattagtggttagagagtgagctattcgttgaaacaattgcgttcttgaaacaattcttgctggtaaaatgtcacattttatgtgactacaggtggaggattggcacataacctaaccagtgggggaaacaattgacctctggatttgtccaagtgtatagtagcatttgcccaatcgaatggtcctggtaaggtgttaatgttgactagaaccaaaggtggaagttgcagggaaactggtttagtacaagggtggacaccaggcagtcatccagaggcccattaaaggccttggaatgtttttccgaaggagaatcactccctcttctctcgcttaaagttttaggggattcatgaacagctgctgtgggatagtttcatgtccctagcaattgtaaagcaactgagggtggcttaaaccagttttagctttagggttagggttactggactaaaatttgagaaattcataaatcttaaggaaatccattgtgagttttcattatgagtgcatccaatgtataatttccatgaccctcccatgcaagtgagcatgtgaatcaggaaacgttacaagaacccaacaaactcaaccactactagacaggcgatcacttccagttagtatgcaactttctgtgtaattttagttaccattaaaatctggatgaccttagtgtaaggaaaaaataccttgaatagtgttaaagatgtacacttggtgtcaggcattgtaacattgataaatctgtgtaaggtgctttttgaaaacttcaaagctgcatcaagtcaagtacaagaaaggccatggctgctaaagctgttgaagatgtgggatggaactgggtcacattggtgttaacagcgttgtgcagagccggcaggatcttggtgtgagcgaacattagtctatttaataaagctgtgtgaatgttgtagaggtgaggatgctcacttgaaaactcactgaagaacacttggccccttgaactaaagtgcttctatcaagttcagtgagaaattccgaattacaagcataggtactagaaaagttttgaaaagcagtatagagcaacataagcacattcataaaattagtgatgtagaaagtgaaatttccacgtatggtcactcccagagaaaaaaaatacgtttatttaccttttttaaaaataggggatttcaggccgggtgaggtggctcacgcctgtaatcccagcactttgggaggcccaggtgggcggatcacctgaggtcaggagttggagggatggcaaatcccatctctacaaaatatacaaaaaaatagctgggtgtgttggcaggcgcctgtaatcccagctactcggaaggctgaggcaggagaatccctggaaccagggatgtggaggttgcagtgagccgagattgtgtaactgcattccagcctgggcaacaagagcaagactccgtatcaggaaaaaaaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacaagatttctattttaattggattcttagggctaataaaataatcagccttagcacttatttatttattttttttgagagggagtctcgctctgttgtccatgctggagtgcagtggcgtgatctcggctcactgcaagctccacctcatgagttcacaccattctcctgcctcagtctcccgagtagctgggactccaggcgccctctacaaagcccgtctaattttttttgtatttttagtagagacagggtttcactgtgttagccaggatggtcttgatctcctgaccttgtgatctgcccgcctcggcctcccaaagtgctgggattataggcttgagccactgctcccggccagcacttatttttataattcttcatgattactgtgttactgtcccatgggccgccagggccagctaggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctagcacgcgctaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggactctagaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgcaagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaccggtccgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgaccggccgctctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctagaggccgccagggccatatttctcaatttttaaatttttcaaaaaaattaatccttaatgtgcatatttttgaattgttaatataactttttgaggtgatgtcttcatgtgtttcaactacttaaaaacttttaaacagtatataataaaaaatcttccaggccactcacacctgtaatcccagcactttgggaggctgaggtgggcagatcacctgagggcaggagttcgagaccagcctggccaatatatatatattcatatattcatatatatatatatattcatatattcatatatatatattcatatattcatatatatatatatatatatatatagcaaaacctcatctctaataaaatacaaaaattagctgagcgtggtgatggatgcctgtagtcccagctactcgggaggctgaggcaggagaatctcttgaacctgggaggtggaggttgcagtgagctgagatggtgccactgccctccagcctgagtgacagagcgagactcggtctccaaaaaaaaacaacaaaaaaatcttccatccttgtctcccatccaccccttccccccagcatgtacttgcagactttatgcatatacagtgagtactgtatatacacaaataataaaaaaatcatatatataatatatgtaattcccctttacatgaaaggtagcacactggtctgtacagtctgtctgcactgtgctatttcactttatatttttatagtttgacagagttctaacatttctttttttttttttttaacagagtcttgttcctgattgttaaattttaaagcatcctaaagtttggtttcacacttgaatgaataccatgtaaggattcacttacatagatgtggttgcctgaatcttaagaataaaataacattgtttgtatttatttaaattagtgttccttttatggtttgcctgaaagcacaacaaaatcctcaccaagatattacaattatgactcccatacaggtaaactgtttagagattggcaagcaccttttaatgaaaggagtcagccagcttagtgtgcagtatttatttctgccggaagagggagcttcagggacagactttggtttagtcatgaagcctccagcactcccaagcggttgtggttgaccaagcaatttatgcttttacctttctacttccagaggcttgtttacttatcagtaagcattaatttagtgtcccctcagatgccttttactttcttcttttctgcctagaataagctgctcttccaattttgcagctacatgtttccaccccagttggaatttctccataacatccattgtagctatccttcaatctacagcctctatttcctgttatagctggtcaggtctaatccctcaaaatactctgtcccctgcttcccttatctgctggccacctttttcccccacatacacactgccatgtcccacccttcactcaagttgttccctgccacctcaacaaatttaagtccataaaatagagtaagtgttcctgactgttaaattttaaagcatcccaaagtctgatttcacactcgaatgaatactatgtacggattcatttacatagatgcggttgcatgagtcttaacaaaaaaataacattatttgtatttattcaaagtactgtcaagatataatgtcaagacctaattcaaaggttccacaaagccttccttgactgcccccaacgaagattatccattttccctgaaatcccattgacttttctattttgtaaggaggctcgtgagactctgtctaaaaacaaaacaaaacaaaaagaaacaatcaaacggcttgcttctgttctttgatctgctagtaagcaaaaattacacatggtgacaggagctatgtgaggctgtcaggttgaatgggaggagtttgggatcctgcttgtggatggttggaagaggctttcgggaaagacagtatttatgtgagacctggaagatgggccttagctttgcagaaggtggagaggcaggaaatagcacgggggccctggggctggaagacttgggcatatttgaggaacagaaaggagaccagcataactgaggtgggaaaagcatgtgaagagatggggctggaggaggccgggagtggtggctcacgcctgtaatcccagcactttgggaggccaaggcaggcggatcatgagctcaggagattgagaccatcctggctaacacggtgaaaccccctctctactaaaaatacaaaaaaaaaaaaaaaaaaaattagctgggcgtggtggcaggagcctgtagtcccagctacctgggaggctgaggcaggagaatggcgtgaacctggaaggctgagcttgcagtgagccgagattgcaccactgcactccagcctgggagacagagagagactccctctcaaaaaaacaaacaaacgaaacaaaacaaaacaaaaattagccaggcgtggtggtatgcacctgtaatcccagctactcgggaggttgaggcaggagaaacgcttgaactcaggaggcggaggttgcagtgagccgagactgcgccactgcactccagcctgggtgacagagggagactccatctcaaaaaaaaaaatttttttttttttacaaacggtgtctccctctgtcgcccaggctggagtgcagtggtgtgatcacagctcactccagcctcaacctccccagctgaagccatcctcttgcctcagcctcctaagtagctgggactacaggcgcgcacctccaggcttggctcttattctttttattgtttttgaaactatagaacctatttttaaaaaatgttttggttgtttttattgctgcttttccttttggggttagaacacaagttttgatgggaaacaggttagaacacattcatctcttcccatagcgatggtcatagaaaaacggggcatatttataaactctcagttgatcttaaaatgtgcaaaagctgccgaactcctgggagtgagctcgagccctgcaggatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 3 - DNA, 1812 bp, synthetic construct
  source 1..1812: mol_type = other DNA
atggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctga

SEQ ID NO: 4 - protein, 603 aa, Homo sapiens
MASASTSKYNSHSLENESIKRTSRDGVNRDLTEAVPRLPGETLITDKEVIYICPFNGPIKGRVYITNYRLYLRSLETDSSLILDVPLGVISRIEKMGGATSRGENSYGLDITCKDMRNLRFALKQEGHSRRDMFEILTRYAFPLAHSLPLFAFLNEEKFNVDGWTVYNPVEEYRRQGLPNHHWRITFINKCYELCDTYPALLVVPYRASDDDLRRVATFRSRNRIPVLSWIHPENKTVIVRCSQPLVGMSGKRNKDDEKYLDVIRETNKQISKLTIYDARPSVNAVANKATGGGYESDDAYHNAELFFLDIHNIHVMRESLKKVKDIVYPNVEESHWLSSLESTHWLEHIKLVLTGAIQVADKVSSGKSSVLVHCSDGWDRTAQLTSLAMLMLDSFYRSIEGFEILVQKEWISFGHKFASRIGHGDKNHTDADRSPIFLQFIDCVWQMSKQFPTAFEFNEQFLIIILDHLYSCRFGTFLFNCESARERQKVTERTVSLWSLINSNKEKFKNPFYTKEINRVLYPVASMRHLELWVNYYIRWNPRIKQQQPNPVEQRYMELLALRDEYIKRLEELQLANSAKLSDPPTSPSSPSQMMPHVQTHF